(19) United States
(12) Patent Application Publication
(10) Pub. No.: US 2002/0058609 A1
(43) Pub. Date: May 16, 2002

(54) COMPOSITIONS AND METHODS USING COMPLEXES OF CALRETICULIN AND ANTIGENIC MOLECULES

(76) Inventors: ELI GILBOA, DURHAM, NC (US); SMITA K. NAIR, DURHAM, NC (US); CHRISTOPHER V. NICCHITTA, DURHAM, NC (US)

Correspondence Address: JENKINS & WILSON, PA, 3100 TOWER BLVD, SUITE 1400, DURHAM, NC, 27707, US

(21) Appl. No.: 09/261,473

(22) Filed: Feb. 26, 1999

(51) Int. Cl. A01N 37/18; A61K 38/00; A61K 39/00; C07K 2/00; C07K 4/00; C07K 5/00; C07K 7/00; C07K 14/00; C07K 16/00; C07K 17/00; C07K 1/00

(52) U.S. Cl. 514/2; 530/300; 530/350

(57) ABSTRACT

A method of eliciting an immune response in a vertebrate subject. The method includes the administration to a vertebrate subject of a composition including an amount of a purified complex including calreticulin bound to an antigenic molecule to elicit an immune response to the antigenic molecule in the vertebrate subject. Therapeutic methods, compositions and kits are also disclosed wherein the elicited immune response is utilized as a treatment for cancer and for infectious diseases.

### GRANT STATEMENT

This workwas supported by NIH grant DK53058. The U.S. Government has certain rights in the invention.

### TECHNICAL FIELD

The present invention relates to compositions and methods pertaining to complexes of endoplasmic reticulum resident peptide binding proteins and antigenic molecules. More particularly, the present invention relates to the use of the endoplasmic reticulum resident peptide binding protein calreticulin in a complex with bound antigen peptides in immunotherapy of cancer and of infectious diseases.

Table of Abbreviations

| | |
|---|---|
| APC | antigen presenting cells |
| BiP | ER hsp70 homolog |
| BMDC | bone marrow-derived dendritic cells |
| CEA | carcinoembryonic antigen(s) |
| CT | computed tomographic |
| CTL | cytotoxic T lymphocyte(s) |
| DC | dendritic cells |
| DMEM | Dulbecco's modified Eagle's medium |
| DTH | delayed-type hypersensitivity |
| ER | endoplasmic reticulum |
| GALT | gut-associated lymphoid tissue |
| gp96/GRP94 | ER paralog of the hsp90 family of chaperones |
| HIV | human immunodeficiency virus |
| HPLC | high pressure liquid chromatography |
| hr | hour(s) |
| hsp(s) | heat shock protein(s) |
| HSV | herpes simplex virus |
| IFN | interferon |
| Ig | immunoglobulin |
| IGF-1 | insulin-like growth factor |
| IgG | immunoglobulin G |
| IL | interleukin |
| MHC | major histocompatability complex |
| min | minute |
| MLTC | mixed lymphocyte tumor cell assay |
| PDI | protein disulfide isomerase |
| PSA | prostate-specific antigen |
| RSV | respiratory syncytial virus |
| RT | room temperature |
| SDS-PAGE | sodium dodecyl sulfate-polyacrylamide gel |
| | electrophoresis |
| TAP | transporter associated with antigen presentation |
| | complex |
| TFA | trifluoroacetic acid |
| TNF | tumor necrosis factor |

### BACKGROUND ART

Calreticulin is an abundant, 46 kDa resident protein of the endoplasmic reticulum (ER) lumen, displays lectin activity and is known to participate in the folding and assembly of nascent glycoproteins. See Nash et al. Mol. Cell. Biochem. 135:71-78 (1994); Hebert et al. EMBO J. 15:2961-2968 (1996); Vassilakos et al. Biochem. 37:3480-3490 (1998); Spiro et al. J. Biol. Chem. 271:11588-11594 (1996); Hebert et al. J. Cell Biol. 139:613-623 (1997). Calreticulin has recently been identified as a component of the major histocompatability complex (MHC) class I/transporter associated with antigen presentation (TAP) complex. See Sadasivan et al. (1996) Cell 5:103-114; Ortmann et al. (1997) Science 277:1306-1309; Solheim et al. (1997) J. Immunol. 158:2236-2241. This protein complex, comprised of the chaperone calreticulin; the TAP transporters TAP1 and TAP2; tapasin; class I heavy chain; and β2microglobulin (β2m), functions in the loading of peptides onto nascent MHC class I molecules (Sadasivan et al. (1996) Cell. 5:103-114; Ortmann et al. (1997) Science 277:1306-1309; Solheim et al. (1997) J. Immunol. 158:2236-2241).

At present, the precise contribution of calreticulin to peptide loading onto class I heavy chain-β2microglobulin dimers remains to be identified. Analysis of the protein-protein interactions preceding peptide loading onto class I molecules suggests that calreticulin plays a role in regulating the association of class I-β2m dimers with TAP, and hence in the regulation of peptide assembly onto nascent class I molecules. See Sadasivan et al. (1996) Cell. 5:103-114; Ortmann et al. (1997) Science. 277:1306-1309; Solheim et al. (1997) J. Immunol. 158:2236-2241; Powis, S. J. (1997) Eur. J. Immunol. 27:2744-2747.

The composite function of the ER lumenal chaperones is generally thought to be limited to the structural maturation of nascent polypeptides. However, the observations that ER chaperones such as GRP94 (gp96), GRP78 (BiP) and protein disulfide isomerase (PDI) display peptide binding activity may portend alternative, or additional roles for these proteins in the regulation of peptide trafficking within the ER. See Spee and Neefjes (1997) Eur. J. Immunol. 27:2441-2449; Blachere et al. (1997) J. Exp. Med. 186:1315-1322; Wearsch and Nicchitta (1997) J. Biol. Chem. 272:5152-5156; Noiva et al. (1991) J. Biol. Chem. 266:19645-19649; Flynn et al. (1989) Science 245:385-390; Lammertetal. (1997) Eur. J. Immunol. 27:1685-1690. ER Hsp90 and GRP94 bind peptides suitable for assembly onto class I molecules (Blachere et al. (1997) J. Exp. Med. 186:1315-1322; Wearsch and Nicchitta (1997) J. Biol. Chem. 272:5152-5156; Suto and Srivastava (1995) Science 269:1585-1588; Arnold et al. (1995) J. Exp. Med. 182:885-889; Nicchitta, C. V. (1998) Curr. Opin. Immunol. 10:103-109). Whether this activity is indicative of a peptide “sink” function, or perhaps is reflective of a more substantive role in peptide/class I assembly reactions remains to be determined.

The potential functional significance of the peptide binding activity is evident, though, in the observations that vaccination of mice with GRP94 can elicit a substantial cellular immune response to components of the bound peptide pool (Blachere et al. (1997) J. Exp. Med. 186:1315-1322; Suto and Srivastava (1995) Science. 269:1585-1588; Arnold et al. (1995) J. Exp. Med. 182:885-889; Nicchitta, C. V. (1998) Curr. Opin. Immunol. 10:103-109; Tamura et al. (1997) Science. 278:117-120). Thus, GRP94, when isolated from a variety of host backgrounds, including tumor cells or cells expressing viral or bacterial proteins, was capable of eliciting substantial CD8+ T cell responses to the parent tumors, as measured in tumor-mass regression studies, as well as known viral and bacterial peptide epitopes, as determined by CTL assay (Blachere et al. (1997) J. Exp. Med. 186:1315-1322; Suto and Srivastava (1995) Science. 269:1585-1588; Arnold et al. (1995) J. Exp. Med. 182:885-889; Tamura et al. (1997) Science 278:117-120).

However, there a great need in the art pertaining to the characterization of the biological role or roles of chaperone proteins. Particularly, the issue of whether other chaperone proteins play a role in the elicitation of immune responses remains unexplored. The characterization of another chaperone protein having such a role would address a long-felt yet continuing need for new and effective therapies for problematic disorders, including a variety of cancers and infectious diseases.

### SUMMARY OF THE INVENTION

In accordance with the present invention, a method of eliciting an immune response against an antigen in a vertebrate subject is provided. The method comprises administering to a vertebrate subject a composition comprising an amount of a purified complex including calreticulin non-covalently bound to an antigenic molecule to elicit an immune response to the antigenic molecule in the vertebrate subject.

Another method of eliciting an immune response in a vertebrate subject is also disclosed herein. The method comprises the step of administering to the vertebrate subject an immunogenic amount of sensitized antigen presenting cells, wherein the antigen presenting cells have been sensitized in vitro with a complex comprising calreticulin non-covalently bound to an antigenic molecule, whereby an immune response to the antigenic molecule is elicited in the vertebrate subject.

Therapeutic methods, preparative methods, compositions and kits are also disclosed wherein an immune response elicited in accordance with the present methods is utilized in the treatment of cancer and of infectious diseases.

Accordingly, it is an object of the present invention to provide an improved method for eliciting an immune response in a vertebrate subject, and preferably, in a human subject.

It is another object of the present invention to provide for the immunotherapy of cancer.

It is still another object of the present invention to provide for the immunotherapy of infectious diseases.

Some of the objects of the invention having been stated hereinabove, other objects will become evident as the description proceeds, when taken in connection with the accompanying Drawings and Examples as best described hereinbelow.

### BRIEF DESCRIPTION OF THE DRAWINGS

FIG. 1 depicts chemical identification of calreticulin-bound peptides.

FIG. 1A depicts digital images of Coomassie Blue-stained 2D SDS-PAGE gels which reflect purity of chaperone fractions. Five (5) μg of purified GRP94 and calreticulin were subject to 2D SDS-PAGE to ascertain purity.

FIG. 1B depicts a digital image of a Coomassie Blue-stained SDS-PAGE gel which reflects extraction of bound peptides. To determine if conditions used for extraction of bound peptides resulted in hydrolysis or degradation of calreticulin, SDS-PAGE analysis of the starting calreticulin (1.5 μg) (lane 1), concentrated pre-extraction (1.5 μg) (lane 2), post-extraction ultra-filtration retentate (15 μg) (lane 3) and the post-extraction filtrate (lane 4) was performed.

FIG. 1C is a graph depicting analytical gel filtration analysis of caireticulin-derived peptide pool. The low molecularweight calreticulin-derived fraction was subject to reductive methylation with [³H] sodium borohydride, fractionated on SEPHADEX™ G-10 to remove unincorporated isotope, concentrated, and analyzed on a Pharmacia SUPERDEX™ peptide column. Sample absorbance at 280 nm was continuously monitored. Fractions were collected and [³H] content determined by liquid scintillation chromatography.

FIG. 1D is a bar graph depicting relative amino acid content of calreticulin-derived peptide fraction. 10.5 nmol of purified calreticulin was extracted; the bound peptide fraction subject to acid hydrolysis in vacuo; and the amino acid content determined by quantitative amino acid analysis. The relative amino acid abundance is presented. Glycine, the most abundant amino acid in the extract, was assigned a value of 1.00. For comparative purposes, the amino acid composition of calreticulin is shown.

FIG. 2 is a set of three graphs depicting immunization with tumor-derived calreticulin or GRP94 elicit tumor-specific CTL responses in vivo. Mice were immunized intravenously twice, at a fourteen day interval, with 10 μg of chaperone protein. Splenocytes were isolated from the immunized mice 10 days after the last immunization and restimulated in vitro with irradiated IFN-γ pretreated F10.9 cells. CTL activity was assayed using F10.9 (H2-K^{b}) cells as targets. EL4 and BALB/3T3 target cells were included as controls for the specificity of the CTL response. ▴=PBS; ●=F10.9 GRP94; ◯=porcine pancreas GRP94; ▪=F10.9 calreticulin; □=porcine pancreas calreticulin.

FIG. 3 is a set of two graphs depicting that the capacity to elicit tumor-specific CTL responses is primarily limited to calreticulin and GRP94. Spleen-derived dendritic cells were pulsed with either B16/F10.9 or mouse spleen-derived ER chaperone proteins in the presence of the cationic lipid DMRIE as described in the Examples. Naive, syngeneic mice were immunized intravenously with 5×10⁵ DC per mouse in 200 μl PBS, two times, at a fourteen day interval. Splenocytes were harvested 10 days post-immunization and restimulated with irradiated F10.9 cells pretreated with IFN-γ. CTL activity was assayed against F10.9 cells and EL4 target cells. ◯=splenic GRP94; □=splenic Erp72; Δ=splenic calreticulin; ▴=F10.9 calreticulin; ●=F10.9 GRP94; •=F10.9 PDI; ▪=F10.9 Erp72, ∘=F10.9 BiP.

FIG. 4 present three graphs which depict the priming of OVA-specific CTL following immunization with E.G7-OVA-chaperone pulsed dendritic cells. GRP94, calreticulin, Hsp90 and Hsp70 were purified from E.G7-OVA and EL4 tumors. Spleen-derived DC were pulsed with the indicated chaperones as described in the Examples. Mice were subjected to a single immunization with 1-2×10⁶ irradiated, chaperone-pulsed DC. Splenocytes were harvested after 7-10 days and restimulated in vitro with irradiated E.G7-OVA. CTL was assayed as described in the Examples. As a control for CTL specificity, parallel assays were performed with F10.9 as target cells. ▪=EL4 GRP94; ▪=E.G7 GRP94; •=E14 calreticulin; ●=E.G7 calreticulin; ▴=EL4 Hsp70; ▴=E.G7 Hsp70; ∘=E14 Hsp90; ◯=E.G7 Hsp90.

FIG. 5 shows that calreticulin-bound OVA peptide gains access to the MHC class I pathway of professional antigen presenting cells and is presented for recognition by class I restricted, OVA-specific CTL.

FIG. 5A is a graph depicting CTL assay data using the OVA-specific CTL line 4G3. Murine bone marrow dendritic cells were generated as described in the Examples. On day 7 of the culture period, nonadherent cells (immature DC) were labeled with europium and pulsed for 48 hr with the indicated source and concentration of calreticulin or OVA peptide. After 2 days in culture, non-adherent cells were harvested as mature dendritic cells, washed, and used as targets. Cells were assayed for peptide SIINFEKL (OVA) presentation on MHC class I by CTL assay using the OVA-specific CTL line 4G3 (Wafts, C. (1997) Annu. Rev. Immunol. 15:821-850). □=OVA peptide (SIINFEKL—1 ng/ml); ×=control peptide (100 ng/ml); ▪=E.G7 calreticulin (1 μg/ml); ▴=E.G7 calreticulin (2.5 μg/ml); ●=E.G7 calreticulin (6.25 μg/ml); ▴=EL4 calreticulin (2.5 μg/ml).

FIG. 5B is a bar graph depicting the incubation of RMA-S cells in the presence of either control peptide (mut-1; FEQNTAQP), or OVA peptide overnight at 37° C. Day 7 bone-marrow derived dendritic cells (immature DC) were pulsed for 48 hr with the indicated source and concentration of calreticulin or OVA peptide. After 2 days in culture, non-adherent cells were harvested as mature dendritic cells, washed, and used as stimulators. The cells were assayed for presentation of class I-restricted OVA peptide to the OVA peptide-specific T cell hybridoma RF3370 (anti-OVA, K^{b}). RMA-S cells were incubated in the presence of either control peptide (mut-1; FEQNTAQP), or OVA peptide overnight at 37° C. In this assay, OVA presentation and recognition is assayed as the stimulation of IL-2 secretion. IL-2 production was measured using ELISA as perthe manufacturers' instructions (Endogen, Cambridge, Mass.).

### DETAILED DESCRIPTION OF THE INVENTION

In accordance with the present invention, methods and compositions for eliciting an immune response in a vertebrate subject are provided. Methods and compositions for the prevention and treatment of infectious diseases and primary and metastatic neoplastic diseases, including, but not limited to, human sarcomas and carcinomas are also provided. In the practice of the prevention and treatment of infectious diseases and cancer, compositions of complexes of the endoplasmic reticulum (ER) resident peptide binding protein calreticulin bound (preferably non-covalently bound) to antigenic molecules, are used to augment the immune response to tumors and infectious agents.

Calreticulin is an endoplasmic reticulum (ER) chaperone protein that displays lectin activity and contributes to the folding pathways for nascent glycoproteins. Calreticulin also participates in the reactions yielding assembly of peptides onto nascent MHC class I molecules. By chemical and immunological criteria, calreticulin is identified herein as a peptide binding protein. Additionally, calreticulin can elicit cytotoxic T lymphocyte (CTL) responses to components of its bound peptide pool.

In an exemplary adoptive immunotherapy method in accordance with the present invention, dendritic cells, pulsed with calreticulin isolated from B16/F10.9 murine melanoma, E.G7-OVA or EL4 thymoma tumors, elicited a cytotoxic T lymphocyte (CTL) response to tumor-derived antigens and to the ovalbumin (OVA) antigen. To evaluate the relative efficacy of calreticulin in eliciting CTL responses, the ER chaperones GRP94/gp96, BiP, ERp72 and protein disulfide isomerase (PDI) were purified in parallel from B16/F10.9, EL4 and E.G7-OVA tumors. The capacity of the proteins to elicit CTL responses was compared. In both the B16/F10.9 and E.G7-OVA model systems models, calreticulin was as or more effective than GRP94/gp96 in eliciting CTL responses. Little to no activity was observed for BiP, ERp72 and PDI. The observed antigenic activity of calreticulin was recapitulated in in vitro experiments where it was observed that pulsing of bone marrow dendritic cells with E.G7-OVA-derived calreticulin elicited sensitivity to lysis by OVA-specific CD8+ T cells. These data identify calreticulin as a peptide binding protein and indicate that calreticulin-bound peptides can be re-presented on dendritic cell class I molecules for recognition by CD8+ T cells.

A. Definitions

While the following terms are believed to have well defined meanings in the art, the following definitions are set forth to facilitate explanation of the invention.

“Antigenic molecule” as used herein refers to the peptides with which calreticulin endogenously associates in vivo (e.g., in infected cells or precancerous or cancerous tissue) as well as exogenous antigens/immunogens (i.e., with which calreticulin is not complexed in vivo) or antigenic/immunogenic fragments and derivatives thereof.

“Adoptive immunotherapy” as used herein refers to refers to a therapeutic approach with particular applicability to cancer in which immune cells with an antitumor reactivity are administered to a tumor-bearing host, with the aim that the cells mediate either directly or indirectly, the regression of an established tumor.

The term “immune system” includes all the cells, tissues, systems, structures and processes, including non-specific and specific categories, that provide a defense against antigenic molecules, including potential pathogens, in a vertebrate subject. As is well known in the art, the non-specific immune system includes phagocytositic cells such as neutrophils, monocytes, tissue macrophages, Kupffer cells, alveolar macrophages, dendritic cells and microglia. The specific immune system refers to the cells and other structures that impart specific immunity within a host. Included among these cells are the lymphocytes, particularly the B cell lymphocytes and the T cell lymphocytes. These cells also include natural killer (NK) cells. Additionally, antibody-producing cells, like B lymphocytes, and the antibodies produced by the antibody-producing cells are also included within the term “immune system”. The term “biological activity” is meant to refer to a molecule having a biological or physiological effect in a vertebrate subject. Adjuvant activity is an example of a biological activity. Activating or inducing production of other biological molecules having adjuvant activity is also a contemplated biological activity.

The term “a biological response modifier” is meant to referto a molecule having the ability to enhance or otherwise modulate a vertebrate subject's response to a particular stimulus, such as presentation of an antigen.

The term “adjuvant activity” is meant to refer to a molecule having the ability to enhance or otherwise modulate the response of a vertebrate subject's immune system to an antigen.

The term “immune response” is meant to refer to any response to an antigen orantigenic determinant by the immune system of a vertebrate subject. Exemplary immune responses include humoral immune responses (e.g. production of antigen-specific antibodies) and cell-mediated immune responses (e.g. lymphocyte proliferation), as defined herein below.

An “immunogenic composition” is meant to refer to a composition that can elicit an immune response. A vaccine is contemplated to fall within the meaning of the term “immunogenic composition”, in accordance with the present invention.

The term “systemic immune response” is meant to refer to an immune response in the lymph node-, spleen-, or gut-associated lymphoid tissues wherein cells, such as B lymphocytes, of the immune system are developed. For example, a systemic immune response can comprise the production of serum IgG's. Further, systemic immune response refers to antigen-specific antibodies circulating in the blood stream and antigen-specific cells in lymphoid tissue in systemic compartments such as the spleen and lymph nodes.

The terms “humoral immunity” or “humoral immune response” are meant to refer to the form of acquired immunity in which antibody molecules are secreted in response to antigenic stimulation.

The terms “cell-mediated immunity” and “cell-mediated immune response” are meant to refer to the immunological defense provided by lymphocytes, such as that defense provided by T cell lymphocytes when they come into close proximity to their victim cells. A cell-mediated immune response also comprises lymphocyte proliferation. When “lymphocyte proliferation” is measured, the ability of lymphocytes to proliferate in response to specific antigen is measured. Lymphocyte proliferation is meant to refer to B cell, T-helper cell or CTL cell proliferation.

The term “CTL response” is meant to refer to the ability of an antigen-specific cell to lyse and kill a cell expressing the specific antigen. As described hereinbelow, standard, art-recognized CTL assays are performed to measure CTL activity.

B. Therapeutic Methods

The methods of the invention comprise methods of eliciting an immune response in a vertebrate subject in which the treatment or prevention of infectious diseases or cancer is desired by administering a composition comprising an effective amount of a complex, wherein the complex preferably comprises calreticulin bound to an antigenic molecule. More preferably, the complex comprises calreticulin non-covalently bound to an antigenic molecule.

The patient treated in the present invention in its many embodiments is desirably a human patient, although it is to be understood that the principles of the invention indicate that the invention is effective with respect to all vertebrate species, including mammals, which are intended to be included in the term “patient”. In this context, a mammal is understood to include any mammalian species in which treatment or prevention of cancer or infectious diseases is desirable, particularly agricultural and domestic mammalian species.

The methods of the present invention are thus particularly contemplated to be useful in the treatment of warm-blooded vertebrates. Therefore, the invention concerns mammals and birds.

More particularly, contemplated is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, contemplated is the treatment of livestock, including, but not limited to, domesticated swine (pigs and hogs), ruminants, horses, poultry, and the like.

In a preferred embodiment, the complex is “autologous” to the vertebrate subject; that is, the complex is isolated from eitherfrom the infected cells orthe cancer cells or precancerous cells of the vertebrate subject (e.g., preferably prepared from infected tissues or tumor biopsies of a vertebrate subject).

Alternatively, the complex is produced in vitro (e.g., wherein a complex with an exogenous antigenic molecule is desired). Alternatively, the calreticulin and/or the antigenic molecule can be isolated from a particular vertebrate subject or from others or by recombinant production methods using a cloned calreticulin originally derived from a particularvertebrate subject orfrom others. Exogenous antigens and fragments and derivatives (both peptide and non-peptide) thereof for use in complexing with calreticulin, can be selected from among those known in the art, as well as those readily identified by standard immunoassays know in the art by the ability to bind antibody or MHC molecules (antigenicity) or generate immune response (immunogenicity). Complexes of calreticulin and antigenic molecules can be isolated from cancer or precancerous tissue of a patient, or from a cancer cell line, or can be produced in vitro (as is necessary in the embodiment in which an exogenous antigen is used as the antigenic molecule).

The invention also provides a method for measuring tumor rejection in vivo in an individual, preferably a human comprising measuring the generation by the individual of MHC Class I-restricted CD8+ cytotoxic T lymphocytes specific to the tumor. Preferably, calreticulin comprises human calreticulin. The immunogenic calreticulin-peptide complexes of the invention may include any complex containing an calreticulin and a peptide that is capable of inducing an immune response in a mammal. The peptides are preferably non-covalently associated with the calreticulin.

Although the calreticulin can be allogeneic to the patient, in a preferred embodiment, the calreticulin are autologous to (derived from) the patient to whom they are administered. The calreticulin and/or antigenic molecules can be purified from natural sources, chemically synthesized, or recombinantly produced. The invention provides methods for determining doses for human cancer immunotherapy by evaluating the optimal dose of calreticulin noncovalently bound to peptide complexes in experimental tumor models and extrapolating the data. Specifically, a scaling factor not exceeding a fifty fold increase over the effective dose estimated in animals, is used as the optimal prescription method for cancer immunotherapy or vaccination in human subjects.

The invention provides combinations of compositions which enhance the immunocompetence of the host individual and elicit specific immunity against infectious agents or specific immunity against preneoplastic and neoplastic cells. The therapeutic regimens and pharmaceutical compositions of the invention are described below. These compositions have the capacity to prevent the onset and progression of infectious diseases and prevent the development of tumor cells and to inhibit the growth and progression of tumor cells, indicating that such compositions can induce specific immunity in infectious diseases and cancer immunotherapy. For example, calreticulin-antigenic molecule complexes can be administered in combination with other complexes, such as gp96/GRP94, and antigenic molecules in accordance with the methods of the present invention.

While it is not the desire of the applicants to be bound by any particular theory of the operation of the methods of the present invention, calreticulin appears to induce an inflammatory reaction at the tumor site and ultimately cause a regression of the tumor burden in the cancer patients treated. Cancers which can be treated with complexes of calreticulin bound to antigenic molecules include, but are not limited to, human sarcomas and carcinomas.

Accordingly, the invention provides methods of preventing and treating cancer in an individual comprising administering a composition which stimulates the immunocompetence of the host individual and elicits specific immunity against the preneoplastic and/or neoplastic cells. As used herein, “preneoplastic” cell refers to a cell which is in transition from a normal to a neoplastic form; and morphological evidence, increasingly supported by molecular biologic studies, indicates that preneoplasia progresses through multiple steps. Non-neoplastic cell growth commonly consists of hyperplasia, metaplasia, or most particularly, dysplasia (for review of such abnormal growth conditions, see Robbins and Angell, 1976, Basic Pathology, 2d Ed., W. B. Saunders Co., Philadelphia, pp. 68-79).

Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. Although preneoplastic lesions may progress to neoplasia, they may also remain stable for long periods and may even regress, particularly if the inciting agent is removed or if the lesion succumbs to an immunological attack by its host.

The therapeutic regimens and pharmaceutical compositions of the invention may be used with additional adjuvants or biological response modifiers including, but not limited to, the cytokines IFN-α, IFN-γ, IL-2, IL-4, IL-6, TNF, or other cytokine affecting immune cells. In accordance with this aspect of the invention, the complexes of the calreticulin and antigenic molecule are administered in combination therapy with one or more of these cytokines.

The invention also contemplates administration of complexes of calreticulin-antigenic molecules to individuals at enhanced risk of cancer due to familial history or environmental risk factors.

C. Dosage Regimens

It was established in experimental tumor models (Blachere et al., 1993, J. Immunotherapy 14:352-356) that the lowest dose of heat shock proteins noncovalently bound to peptide complexes which produced tumor regression in mice was between 10 and 25 microgram/mouse weighing 20-25 g which is equal to 25 mg/25 g=1 mg/kg. Conventional methods extrapolate to human dosages based on body weight and surface area. For example, conventional methods of extrapolating human dosage based on body weight can be carried out as follows: since the conversion factor for converting the mouse dosage to human dosage is Dose Human per kg=Dose Mouse per kg×12 (See Freireich et al. (1966) Cancer Chemotherap. Rep. 50:219-244), the effective dose of calreticulin-peptide complexes in humans weighing 70 kg should be 1 mg/kg÷12×70, i.e., about 6 mg (5.8 mg).

Drug doses are also given in milligrams per square meter of body surface area because this method rather than body weight achieves a good correlation to certain metabolic and excretionary functions (Shirkey, H. C., 1965, JAMA 193:443). Moreover, body surface area can be used as a common denominator for drug dosage in adults and children as well as in different animal species as described by Freireich et al. (1966) Cancer Chemotherap. Rep. 50:219-244). Briefly, to express a mg/kg dose in any given species as the equivalent mg/sq m dose, multiply the dose by the appropriate km factor. In adult human, 100 mg/kg is equivalent to 100 mg/kgx37 kg/sq m=3700 mg/sq m.

PCT Publications WO 95/24923; WO 97/10000; WO 97/10002; and WO 98/34641, as well as U.S. Pat. Nos. 5,750,119; 5,830,464; and U.S. Pat. No. 5,837,251, each provide dosages of the purified complexes of heat shock proteins and antigenic molecules, and the entire contents of each of these documents are herein incorporated by reference. Briefly, and as applied to the present invention, an amount of calreticulin-antigenic molecule complexes is administered that is in the range of about 10 microgram to about 600 micrograms for a human patient, the preferred human dosage being the same as used in a 25 g mouse, i.e., in the range of 10-100 micrograms. The dosage for calreticulin-peptide complexes in a human patient provided by the present invention is in the range of about 50 to 5,000 micrograms, the preferred dosage being 100 micrograms.

In a series of preferred and more preferred embodiments, the calreticulin-peptide complex is administered in an amount of less than about 50 micrograms. In this case, the calreticulin-peptide complex is preferably administered in an amount of ranging from about 5 to about 49 micrograms.

Optionally, the calreticulin-peptide complex is administered in an amount of less than about 10 micrograms. In this case, the calreticulin-peptide complex is preferably administered in an amount ranging from about 0.1 to about 9.0 micrograms. More preferably, the calreticulin-peptide complexes is administered in an amount ranging from about 0.5 to about 2.0 micrograms.

The doses recited above are preferably given once weekly for a period of about 4-6 weeks, and the mode or site of administration is preferably varied with each administration. In a preferred example, subcutaneous administrations are given, with each site of administration varied sequentially. For example, half the dose may be given in one site and the other half on an other site on the same day.

Alternatively, the mode of administration is sequentially varied. For example, weekly injections are given in sequence subcutaneously, intramuscularly, intravenously or intraperitoneally. After 4-6 weeks, further injections are preferably given at two-week intervals over a period of time of one month. Later injections may be given monthly. The pace of later injections may be modified, depending upon the patient's clinical progress and responsiveness to the immunotherapy.

D. Therapeutic Compositions for Immune Responses to Cancer

Compositions comprising calreticulin bound (preferably non-covalently bound) to antigenic molecules are contemplated in accordance with the present invention for administration to elicit an effective specific immune response to the complexed antigenic molecules (and preferably not to the calreticulin). In a preferred embodiment, non-covalent complexes of calreticulin with peptides are prepared and purified postoperatively from tumor cells obtained from the cancer patient.

In accordance with the methods described herein, immunogenic or antigenic peptides that are endogenously complexed to calreticulin or MHC antigens can be used as antigenic molecules. For example, such peptides may be prepared that stimulate cytotoxic T cell responses against different tumor antigens (e.g., tyrosinase, gp100, melan-A, gp75, mucins, etc.) and viral proteins including, but not limited to, proteins of immunodeficiency virus type I (HIV-I), human immunodeficiency virus type II (HIV-II), hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus (RSV), papilloma virus, papova virus, cytomegalovirus, echinovirus, arbovirus, huntavirus, coxsackie virus, mumps virus, measles virus, rubella virus and polio virus. In the embodiment wherein the antigenic molecules are peptides noncovalently complexed to calreticulin in vivo, the complexes can be isolated from cells, or alternatively, produced in vitro from purified preparations each of calreticulin and antigenic molecules.

In another specific embodiment, antigens of cancers (e.g., tumors) or infectious agents (e.g., viral antigen, bacterial antigens, etc.) can be obtained by purification from natural sources, by chemical synthesis, or recombinantly, and, through in vitro procedures such as those described below, complexed to calreticulin.

In an embodiment wherein the calreticulin-antigenic molecule complex to be used is a complex that is produced in vivo in cells, exemplary purification procedures such as described in the Examples presented below can be employed. Alternatively, in an embodiment wherein one wishes to use antigenic molecules by complexing to calreticulin in vitro, calreticulin can be purified for such use from the endogenous calreticulin-peptide complexes in low pH (or chemically synthesized or recombinantly produced). The protocols described herein may be used to isolate calreticulin-peptide complexes, or the calreticulin alone, from any eukaryotic cells. For example, tissues, isolated cells, or immortalized eukaryote cell lines infected with a preselected intracellular pathogen, tumor cells or tumor cell lines may be used.

E. Infectious Diseases

In an alternative embodiment wherein it is desired to treat a patient having an infectious disease the above-described methods are used to isolate calreticulin-peptide complexes from cells infected with an infectious organism, e.g., of a cell line or from a patient. Such infectious organisms include but are not limited to, viruses, bacteria, protozoa, fungi, and parasites as described in detail hereinbelow.

F. Isolation of Antigenic/Immunogenic Components

It has been found that antigenic peptides and/or components can be eluted from calreticulin-complexes under low pH conditions. These experimental conditions may be used to isolate peptides and/or antigenic components from cells which may contain potentially useful antigenic determinants. Once isolated, the amino acid sequence of each antigenic peptide may be determined using conventional amino acid sequencing methodologies. Such antigenic molecules can then be produced by chemical synthesis or recombinant methods; purified; and complexed to calreticulin in vitro. Additionally, antigenic peptide sequences can be obtained by mass spectrometry using, but not limited to, electrospray and MALDI-TOF instrumentation, coupled with quadrapole detection and CAD-based sequencing.

Similarly, it has been found that potentially immunogenic peptides may be eluted from MHC-peptide complexes using techniques well know in the art (Falk, K. et al., 1990 Nature 348:248-251; Elliott, T., et al., 1990, Nature 348:195-197; Falk, K., et al., 1991, Nature 351:290-296). Thus, potentially immunogenic or antigenic peptides may be isolated from either endogenous calreticulin-peptide complexes orendogenous MHC-peptide complexes for use subsequently as antigenic molecules, by complexing in vitro to calreticulin. Exemplary protocols for isolating peptides and/or antigenic components from either of the these complexes are set forth in the Examples and are presented below.

G. Peptides From Calretculin-peptide Complexes

Several methods may be used to elute the peptide from a calreticulin-peptide complex. The approaches involve incubating the calreticulin-peptide complex in a low pH buffer and/or in guanidinium/HCl (3-6 M), 0.1-1% TFA or acetic acid. Briefly, the complex of interest is centrifuged through a CENTRICON™ 10 assembly (Millipore) to remove any low molecular weight material loosely associated with the complex. The large molecular weight fraction may be removed and analyzed by SDS-PAGE while the low molecular weight material is fractionated by microbore HPLC, with a flow rate of 0.5 ml/min, with monitoring at 210/220 nm.

In the low pH protocol, acetic acid or trifluoroacetic acid (TFA) is added to the calreticulin-peptide complex to give a final concentration of 10% (vol/vol) and the mixture incubated at room temperature, or in a boiling water bath, or any temperature in between, for 10 minutes (See Van Bleek et al. (1990) Nature 348:213-216; and Li et al. (1993) EMBO Journal 12:3143-3151).

The resulting samples are centrifuged through a CENTRICON™ 10 assembly as mentioned previously. The high and low molecular weight fractions are recovered. The remaining large molecular weight calreticulin-peptide complexes can be reincubated with guanidinium or low pH to remove any remaining peptides. The resulting lower molecular weight fractions are pooled, concentrated by evaporation and dissolved in 0. 1% trifluoroacetic acid (TFA). The dissolved material is fractionated by microbore HPLC, with a flow rate of 0.5 ml/min. The elution of the peptides can be monitored by OD210/220 nm and the fractions containing the peptides collected.

H. Peptides from MHC-peptide Complexes

The isolation of potentially immunogenic peptides from MHC molecules is well known in the art and so is not described in detail herein. See Falk et al. (1990) Nature 348:248-251; Rotzsche et al. (1990) Nature 348:252-254; Elliott et al. (1990) Nature 348:191-197; Falk et al. (1991) Nature 351:290-296; Demotz et al. (1989) Nature 343:682-684; Rotzsche et al. (1990) Science 249:283-287), the disclosures of which are incorporated herein by reference. Briefly, MHC-peptide complexes may be isolated by a conventional immunoaffinity procedure. The peptides then may be eluted from the MHC-peptide complex by incubating the complexes in the presence of about 0.1% TFA in acetonitrile. The eluted peptides may be fractionated and purified by HPLC as described above.

The amino acid sequences of the eluted peptides may be determined either by manual or automated amino acid sequencing techniques well known in the art. Once the amino acid sequence of a potentially protective peptide has been determined the peptide may be synthesized in any desired amount using conventional peptide synthesis or other protocols well known in the art.

A contemplated peptide, also referred to herein as a subject peptide, can be synthesized by any of the techniques that are known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. An excellent summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol.2, p.46, Academic Press (New York), 1983; Merrifield, Adv Enzymol, 32:221-96,1969; Fields et al., Int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized foramino acids containing a reactive side group such as lysine.

Using a solid phase synthesis as exemplary, the protected orderivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable forforming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

The resultant linear polypeptides prepared for example as described above may be reacted to form their corresponding cyclic peptides. An exemplary method for cyclizing peptides is described by Zimmer et al., Peptides 1992, pp. 393-394, ESCOM Science Publishers, B. V., 1993. Typically, tertbutoxycarbonyl protected peptide methyl ester is dissolved in methanol and sodium hydroxide solution are added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxytermini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Purification of the resulting peptides is accomplished using conventional procedures, such as preparative HPLC using gel permeation, partition and/or ion exchange chromatography. The choice of appropriate matrices and buffers are well known in the art and so are not described in detail herein.

I. Exocienous Anticienic Molecules.

Antigens or antigenic portions thereof can be selected for use as antigenic molecules, for complexing to calreticulin, from among those known in the art or determined by immunoassay to be able to bind to antibody or MHC molecules (antigenicity) or generate immune response (immunogenicity). To determine immunogenicity or antigenicity by detecting binding to antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immuno-electrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are envisioned for use. In one embodiment for detecting immunogenicity, T cell-mediated responses can be assayed by standard methods, e.g., in vitro cytoxicity assays or in vivo delayed-type hypersensitivity assays.

Potentially useful antigens or derivatives thereof for use as antigenic molecules can also be identified by various criteria, such as the antigen's involvement in neutralization of a pathogen's infectivity (wherein it is desired to treat or prevent infection by such a pathogen) (Norrby, 1985, Summary, in Vaccines 85, Lerner, et al. (eds.), Cold Spring Harbor Laboratory, Cold Spring Harbor, N.Y., pp. 388-389), type or group specificity, recognition by patients' antisera or immune cells, and/or the demonstration of protective effects of antisera or immune cells specificforthe antigen. In addition, where it is desired to treat or prevent a disease caused by a pathogen, the antigen's encoded epitope should preferably display a small or no degree of antigenic variation in time or amongst different isolates of the same pathogen.

Preferably, where it is desired to treat or prevent cancer, known tumor-specific antigens or fragments or derivatives thereof are used. For example, such tumor specific or tumor-associated antigens include but are not limited to KS 1/4 pan-carcinoma antigen (Perez and Walker (1990) J. Immunol. 142:3662-3667; Bumal (1988) Hybridoma 7(4):407-415); ovarian carcinoma antigen (CA125) (Yu et al. (1991) Cancer Res. 51 (2):468-475); prostatic acid phosphate (Tailer et al. (1990) Nucl. Acids Res. 18(16):4928); prostate specific antigen (Henttu and Vihko (1989) Biochem. Biophys. Res. Comm. 160(2):903-910; Israeli et al. (1993) Cancer Res. 53:227-230); melanoma-associated antigen p97 (Estin et al. (1989) J. Natl. Cancer Inst. 81 (6):445-446); melanoma antigen gp75 (Vijayasardahl et al. (1990) J. Exp. Med. 171(4): 1375-1380); high molecularweight melanoma antigen (Natali et al. (1987) Cancer 59:55-63) and prostate specific membrane antigen.

In a specific embodiment, an antigen or fragment or derivative thereof specific to a certain tumor is selected for complexing to calreticulin and subsequent administration to a patient having that tumor. Preferably, where it is desired to treat or prevent viral diseases, molecules comprising epitopes of known viruses are used. For example, such antigenic epitopes may be prepared from viruses including, but not limited to, hepatitis type A hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus (RSV), papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II). Preferably, where it is desired to treat or prevent bacterial infections, molecules comprising epitopes of known bacteria are used. For example, such antigenic epitopes may be prepared from bacteria including, but not limited to, Mycobacteria, Rickettsia, Mycoplasma, Neisseria and Legionella.

Preferably, where it is desired to treat or prevent protozoal infectious, molecules comprising epitopes of known protozoa are used. For example, such antigenic epitopes may be prepared from protozoa including, but not limited to, Leishmania, Kokzidioa, and Trypanosoma. Preferably, where it is desired to treat or prevent parasitic infectious, molecules comprising epitopes of known parasites are used. For example, such antigenic epitopes may be from parasites including, but not limited to, Chlamydia and Rickettsia.

J. In vitro Production of Calreticulin-antigenic Molecule Complexes.

In an embodiment in which complexes of calreticulin and the peptides with which they are endogenously associated in vivo are not employed, complexes of calreticulin to antigenic molecules are produced in vitro. As will be appreciated by those skilled in the art, the peptides either isolated by the aforementioned procedures or chemically synthesized or recombinantly produced may be reconstituted with a variety of naturally purified or recombinant calreticulins in vitro to generate immunogenic non-covalent calreticulin-antigenic molecule complexes. Alternatively, exogenous antigens or antigeniclimmunogenic fragments or derivatives thereof can be noncovalently complexed to calreticulins for use in the immunotherapeutic or prophylactic vaccines of the invention. A preferred, exemplary protocol for noncovalently complexing a calreticulin and an antigenic molecule in vitro is discussed in the Examples presented below.

The antigenic molecules (1 μg) and the calreticulin (9 μg) are admixed to give an approximately 5 antigenic molecule:1 calreticulin molar ratio. Then, the mixture is incubated for 15 minutes to 3 hours at 40 to 45° C. in a suitable binding buffer such as one containing 20 mM sodium phosphate, pH 7.2, 350 mM NaCl, 3 mM MgCl2 and 1 mM phenyl methyl sulfonyl fluoride (PMSF). The preparations are centrifuged through CENTRICON™ 10 assembly (Millipore) to remove any unbound peptide. The association of the peptides with the calreticulins can be assayed by SDS-PAGE. This is a preferred method for in vitro complexing of peptides isolated from MHC-peptide complexes of peptides disassociated from endogenous calreticulin-peptide complexes.

Following complexing, the immunogenic calreticulin-antigenic molecule complexes can optionally be assayed in vitro using for example the mixed lymphocyte tumor cell assay (MLTC) described below. Once immunogenic complexes have been isolated they can be optionally characterized further in animal models using the preferred administration protocols and excipients discussed below.

K. Determination of Immunogenicity of Calreticulin-peptide Complexes

The purified calreticulin-antigenic molecule complexes can be assayed for immunogenicity using the mixed lymphocyte tumor culture assay (MLTC) well known in the art. By way of example but not limitation, the following procedure can be used. Briefly, mice are injected subcutaneously with the candidate calreticulin-antigenic molecule complexes. Other mice are injected with either other calreticulin peptide complexes or whole infected cells which act as positive controls for the assay. The mice are injected twice, 7-10 days apart. Ten days after the last immunization, the spleens are removed and the lymphocytes released. The released lymphocytes may be restimulated subsequently in vitro by the addition of dead cells that expressed the complex of interest.

For example, 8×10⁶ immune spleen cells may be stimulated with 4×10⁴ mitomycin C treated or γ-irradiated (5-10,000 rads) infected cells (or cells transfected with an appropriate gene, as the case may be) in 3 ml RPMI medium containing 10% fetal calf serum. In certain cases 33% secondary mixed lymphocyte culture supernatant may be included in the culture medium as a source of T cell growth factors, such as is described by Glasebrook et al. (1980) J. Exp. Med. 151:876. To test the primary cytotoxic T cell response after immunization, spleen cells may be cultured without stimulation. In some experiments spleen cells of the immunized mice may also be restimulated with antigenically distinct cells, to determine the specificity of the cytotoxic T cell response.

Six days later the cultures are tested for cytotoxicity in a 4 hour ⁵1Cr-release assay as is described by Palladino et al. (1987) Cancer Res. 47:5074-5079 and Blachere et al. (1993) J. Immunotherapy 14:352-356. In this assay, the mixed lymphocyte culture is added to a target cell suspension to give different effector:target (E:T) ratios (usually 1:1 to 40:1). The target cells are prelabelled by incubating 1×10⁶ target cells in culture medium containing 200 mCi ⁵¹Cr/ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelleted by centrifugation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade a concentrated hybridoma supernatant derived from K-44 hybridoma cells (an anti-MHC class I hybridoma) is added to the test samples to a final concentration of 12.5%.

L. Formulation

Calreticulin-antigenic molecule complexes of the invention may be formulated into pharmaceutical preparations for administration to mammals for treatment or prevention of cancer or infectious diseases. Compositions comprising a compound of the invention formulated in a compatible pharmaceutical carrier may be prepared, packaged, and labeled for treatment of the indicated tumor, such as human sarcomas and carcinomas.

Exemplary human sarcomas and carcinomas include, but are not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenströom's macroglobulinemia, and heavy chain disease. Alternatively, it can be labeled for treatment of the appropriate infectious disease. Alternatively, pharmaceutical compositions may be formulated for treatment of appropriate infectious diseases.

If the complex is water-soluble, then it may be formulated in an appropriate buffer, for example, phosphate buffered saline or other physiologically compatible solutions. Alternatively, if the resulting complex has poor solubility in aqueous solvents, then it may be formulated with a non-ionic surfactant, such as TWEEN™, or polyethylene glycol. Thus, the compounds and their physiologically acceptable solvates may be formulated for administration by inhalation or insufflation (either through the mouth or the nose) or oral, buccal, parenteral, rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, orfractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); orwetting agents (e.g., sodium lauryl sulphate). The tablets may be coated by methods well-known in the art. Preparations for oral administration may be suitably formulated to give controlled release of the active compound.

For buccal administration, the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, for example, in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying outthe therapeutic regimens of the invention. Such kits comprise in one or more containers therapeutically or prophylactically effective amounts of the calreticulin-antigenic molecule complexes in pharmaceutically acceptable form. The calreticulin-antigenic molecule complex in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the complex, and/or a packaged alcohol pad. Instructions are optionally included for administration of calreticulin-antigenic molecule complexes by a clinician or by the patient.

M. Target Infectious Diseases

Infectious diseases that can be treated or prevented by the methods of the present invention are caused by infectious agents including, but not limited to, viruses, bacteria, fungi, protozoa and parasites.

Viral diseases that can be treated or prevented by the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus (RSV), papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

Bacterial diseases that can be treated or prevented by the methods of the present invention are caused by bacteria including, but not limited to, Mycobacteria, Rickettsia, Mycoplasma, Neisseria and Legionella.

Protozoal diseases that can be treated or prevented by the methods of the present invention are caused by protozoa including, but not limited to, Leishmania, Kokzidioa, and Trypanosoma.

Parasitic diseases that can be treated or prevented by the methods of the present invention are caused by parasites including, but not limited to, Chlamydia and Rickettsia.

N. Target Cancers.

Cancers that can be treated or prevented by the methods of the present invention include, but not limited to human sarcomas and carcinomas, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenströom's macroglobulinemia, and heavy chain disease.

In a specific embodiment the cancer is metastatic. In another specific embodiment, the patient having a cancer is immunosuppressed by reason of having undergone anti-cancer therapy (e.g., chemotherapy radiation) prior to administration of the calreticulin-antigenic molecule complexes of the invention.

O. Combination With Adoptive Immunotherapy

Adoptive immunotherapy refers to a therapeutic approach for treating cancer or infectious diseases in which immune cells are administered to a host with the aim that the cells mediate either directly or indirectly specific immunity to tumor cells and/or antigenic components or regression of the tumor or treatment of infectious diseases, as the case may be. In accordance with the methods described herein, APC are sensitized with calreticulin preferably noncovalently complexed with antigenic (or immunogenic) molecules and used in adoptive immunotherapy.

According to the invention, therapy by administration of calreticulin-peptide complexes, using any desired route of administration, is combined with adoptive immunotherapy using APC sensitized with calreticulin-antigenic molecule complexes. As described herein, the calreticulin-peptide complex-sensitized APC can be administered concurrently with calreticulin-peptide complexes, or before or after administration of calreticulin-peptide complexes. Furthermore, the mode of administration can be varied, including but not limited to, e.g., subcutaneously, intravenously, intraperitoneally, intramuscularly, intradermally or mucosally.

P. Obtaining Macrophages and Antigen-presenting Cells

The antigen-presenting cells, including but not limited to macrophages, dendritic cells and B-cells, are preferably obtained by production in vitro from stem and progenitor cells from human peripheral blood or bone marrow as described by Inaba (1992) J. Exp. Med. 176:1693-1702.

APC can be obtained by any of various methods known in the art. In a preferred aspect human macrophages are used, obtained from human blood cells. By way of example but not limitation, macrophages can be obtained as follows: mononuclear cells are isolated from peripheral blood of a patient (preferably the patient to be treated), by Ficoll-Hypaque gradient centrifugation and are seeded on tissue culture dishes which are pre-coated with the patient's own serum or with other AB+ human serum. The cells are incubated at 37° C. for 1 hr, then non-adherent cells are removed by pipetting. To the adherent cells left in the dish, is added cold (4° C.) 1 mM EDTA in phosphate-buffered saline and the dishes are left at room temperature for 15 minutes. The cells are harvested, washed with RPMI buffer and suspended in RPMI buffer. Increased numbers of macrophages may be obtained by incubating at 37° C. with macrophage-colony stimulating factor (M-CSF); increased numbers of dendritic cells may be obtained by incubating with granulocyte-macrophage-colony stimulating factor (GM-CSF) as described in detail by Inaba, K., et al., 1992, J. Exp. Med. 176:1693-1702.

Q. Sensitization of Macrophages and Antigen Presenting Cells With Calreticulin-peptide Complexes

APC are sensitized with calreticulin (preferably noncovalently) bound to antigenic molecules by incubating the cells in vitro with the complexes. The APC are sensitized with complexes of calreticulin and antigenic molecules preferably by incubating in vitro with the calreticulin-complex at 37° C. for 15 minutes to 24 hours. Byway of example but not limitation, 4×10⁷ macrophages can be incubated with 10 microgram calreticulin-peptide complexes per ml or 100 microgram calreticulin-peptide complexes per ml at 37° C. for 15 minutes-24 hours in 1 ml plain RPMI medium. The cells are washed three times and resuspended in a physiological medium preferably sterile, at a convenient concentration (e.g., 1×10⁷/ml) for injection in a patient. Preferably, the patient into which the sensitized APCs are injected is the patient from which the APC were originally isolated (autologous embodiment).

Optionally, the ability of sensitized APC to stimulate, for example, the antigen-specific, class I-restricted cytotoxic T-lymphocytes (CTL) can be monitored by their ability to stimulate CTLs to release tumor necrosis factor, and by their ability to act as targets of such CTLs.

R. Reinfusion of Sensitized APC

The calreticulin-antigenic molecule-sensitized APC are reinfused into the patient systemically, preferably intravenously, by conventional clinical procedures. These activated cells are reinfused, preferentially by systemic administration into the autologous patient. Patients generally receive from about 10⁶ to about 10¹² sensitized macrophages, depending on the condition of the patient. In some regimens, patients may optionally receive in addition a suitable dosage of a biological response modifier including but not limited to the cytokines IFN-α, IFN-γ, IL-2, IL-4, IL-6, TNF or other cytokine growth factor.

S. Autologous Embodiment.

The specific immunogenicity of calreticulin derives not from calreticulin per se, but from the peptides bound to them. In a preferred embodiment of the invention directed to the use of autologous complexes of calreticulin-peptides as cancer vaccines, two of the most intractable hurdles to cancer immunotherapy are circumvented. First is the possibility that human cancers, like cancers of experimental animals, are antigenically distinct. In an embodiment of the present invention, calreticulin chaperone antigenic peptides of the cancer cells from which they are derived and circumvent this hurdle.

Second, most current approaches to cancer immunotherapy focus on determining the CTL-recognized epitopes of cancer cell lines. This approach requires the availability of cell lines and CTLs against cancers. These reagents are unavailable for an overwhelming proportion of human cancers. In an embodiment of the present invention directed to autologous complexes of calreticulin and peptides, cancer immunotherapy does not depend on the availability of cell lines or CTLs nor does it require definition of the antigenic epitopes of cancer cells. These advantages make autologous calreticulin noncovalently bound to peptide complexes attractive and novel immunogens against cancer.

T. Prevention and Treatment of Primary and Metastatic Neoplastic Diseases.

There are many reasons why immunotherapy as provided bythe present invention is desired for use in cancer patients. First, if cancer patients are immunosuppressed and surgery, with anesthesia, and subsequent chemotherapy, may worsen the immunosuppression, then with appropriate immunotherapy in the preoperative period, this immunosuppression may be prevented or reversed. This could lead to fewer infectious complications and to accelerated wound healing. Second, tumor bulk is minimal following surgery and immunotherapy is most likely to be effective in this situation. A third reason is the possibility that tumor cells are shed into the circulation at surgery and effective immunotherapy applied at this time can eliminate these cells.

The preventive and therapeutic methods of the invention are directed at enhancing the immunocompetence of the cancer patient either before surgery, at or after surgery, and to induce tumor-specific immunity to cancer cells, with the objective being inhibition of cancer, and with the ultimate clinical objective being total cancer regression and eradication.

U. Monitoring of Effects During Cancer Prevention and Immunotherapy with Calreticulin-peptide Complexes.

The effect of immunotherapy with calreticulin-antigenic molecule complexes on development and progression of neoplastic diseases can be monitored by any methods known to one skilled in the art, including but not limited to measuring: 1) delayed hypersensitivity as an assessment of cellular immunity; 2) activity of cytolytic T-lymphocytes in vitro; 3) levels of tumor specific antigens, e.g., carcinoembryonic (CEA) antigens; 4) changes in the morphology of tumors using techniques such as a computed tomographic (CT) scan; 5) changes in levels of putative biomarkers of risk for a particular cancer in individuals at high risk, and 6) changes in the morphology of tumors using a sonogram.

Delayed Hypersensitivity Skin Test.

Delayed hypersensitivity skin tests are of great value in the overall immunocompetence and cellular immunity to an antigen. Inability to react to a battery of common skin antigens is termed anergy (Sato et al. (1995) Clin. Immunol. Pathol 74:35-43). Proper technique of skin testing requires that the antigens be stored sterile at 4° C., protected from light and reconstituted shortly before use. A 25- or 27-gauge needle ensures intradermal, rather than subcutaneous, administration of antigen. Twenty-four and forty-eight hours after intradermal administration of the antigen, the largest dimensions of both erythema and induration are measured with a ruler. Hypoactivity to any given antigen or group of antigens is confirmed by testing with higher concentrations of antigen or, in ambiguous circumstances, by a repeat test with an intermediate concentration.

Activity of Cytolytic T-lymphocytes In vitro.

8×10⁶ peripheral blood derived T lymphocytes isolated by the Ficoll-Hypaque centrifugation gradient technique, are restimulated with 4×10⁴ mitomycin C treated tumor cells in 3 ml RPMI medium containing 10% fetal calf serum. In some experiments, 33% secondary mixed lymphocyte culture supernatant or IL-2, is included in the culture medium as a source of T cell growth factors.

In order to measure the primary response of cytolytic T-lymphocytes after immunization, T cells are cultured without the stimulator tumor cells. In other experiments, T cells are restimulated with antigenically distinct cells. After six days, the cultures are tested for cytotoxity in a 4 hour ⁵¹Cr-release assay. The spontaneous ⁵¹Cr-release of the targets should reach a level less than 20%. For the anti-MHC class I blocking activity, a tenfold concentrated supernatant of W6/32 hybridoma is added to the test at a final concentration of about 12.5% (Heike et al. (199) J. Immunotherapy 15:165-174).

Levels of Tumor Specific Antigens.

Although it may not be possible to detect unique tumor antigens on all tumors, many tumors display antigens that distinguish them from normal cells. Monoclonal antibody reagents have permitted the isolation and biochemical characterization of the antigens and have been invaluable diagnostically for distinction of transformed from nontransformed cells and for definition of the cell lineage of transformed cells. The best-characterized human tumor-associated antigens are the oncofetal antigens. These antigens are expressed during embryogenesis, but are absent or very difficult to detect in normal adult tissue. The prototype antigen is carcinoembryonic antigen (CEA), a glycoprotein found on fetal gut an human colon cancer cells, but not on normal adult colon cells. Since CEA is shed from colon carcinoma cells and found in the serum, it was originally thought that the presence of this antigen in the serum could be used to screen patients for colon cancer. However, patients with other tumors, such as pancreatic and breast cancer, also have elevated serum levels of CEA. Therefore, monitoring the fall and rise of CEA levels in cancer patients undergoing therapy has proven useful for predicting tumor progression and responses to treatment.

Several other oncofetal antigens have been useful for diagnosing and monitoring human tumors, e.g., alpha-fetoprotein, an alpha-globulin normally secreted by fetal liver and yolk sac cells, is found in the serum of patients with liver and germinal cell tumors and can be used as a matter of disease status.

Computed Tomographic (CT) Scan.

CT remains the choice of techniques for the accurate staging of cancers. CT has proved more sensitive and specific than any other imaging techniques forthe detection of metastases.

Measurement of Putative Biomarkers.

The levels of a putative biomarker for risk of a specific cancer are measured to monitor the effect of calreticulin noncovalently bound to peptide complexes. For example, in individuals at enhanced risk for prostate cancer, serum prostate-specific antigen (PSA) is measured by the procedure described by Brawer et al. (1992) J. Urol. 147:841-845 and Catalona et al. (1993) JAMA 270:948-958; or in individuals at risk for colorectal cancer CEA is measured as described above; and in individuals at enhanced risk for breast cancer, 16-a-hydroxylation of estradiol is measured by the procedure described by Schneider et al. (1982) Proc. Natl. Acad. Sci. USA 79:3047-3051. The references cited above are incorporated by reference herein in their entirety.

Sonogram.

A Sonogram remains an alternative choice of technique for the accurate staging of cancers.

The following Examples have been included to illustrate preferred modes of the invention. Certain aspects of the following Examples are described in terms of techniques and procedures found or contemplated by the present inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### EXAMPLES

The Examples describe the investigation of whether calreticulin displays in vivo interactions with peptides. The investigation was performed by direct biochemical analysis of acid extracted, tissue-derived calreticulin and by the capacity of calreticulin purified from B16/F10.9, EI-4 and E.G7-OVA tumors to elicit specific CTL responses. Direct evidence supporting the existence of a calreticulin-bound peptide fraction was obtained. In addition, vaccination of mice with dendritic cells pulsed with B16/F10. 9, EL4 or E.G7-OVA-derived calreticulin, was observed to elicit CTL responses to undefined B16/F10.9 and EL4 antigens as well as to the immunodominant OVA peptide epitope, SIINFEKL, (E.G7-OVA). Lastly, bone marrow-derived dendritic cells (BMDC) pulsed in vitro with E.G7-OVA-calreticulin presented tumor-specific peptides in association with class I molecules and were targeted for lysis by the OVA-specific CTL line, 4G3.

### Materials and Methods Used in Examples

Mice.

5-6 weeks old female C57BL/6 mice (H-2b) and SCID mice were obtained from the Charles River Laboratories, Raleigh, North Carolina. In conducting the experiments described herein, applicants adhered to the “Guide for the Care and Use of Laboratory Animals” as proposed by the committee on care of Laboratory Animal Resources Commission on Life Sciences, National Research Council. The facilities are fully accredited by the American Association for Accreditation of Laboratory Animal Care.

Cell Lines.

Cell lines used were EL4 (C57BL/6, H-2b, thymoma), E.G7-OVA (EL4 cells transfected with the OVA cDNA), RMA-S cells (Rauscher leukemia virus-induced T cell lymphoma RBL-5 of C57BL/6 (H-2b) origin) and B16/F10.9 (F10.9) melanoma. Cells were maintained in DMEM supplemented with 10% heat-inactivated FCS (Gibco, Grand Island, N.Y.), 2 mM glutamine, 100 U/ml penicillin and 100 μg/ml streptomycin. E.G7-OVA cells were grown in medium containing 400 μg/ml G418 (Gibco, Grand Island, N.Y.). T-cell hybridoma RF3370 (H-2K^{b}-restricted, OVA-specific) were maintained in RPMI 1640 (Gibco, Grand Island, N.Y.) supplemented with 10% heat-inactivated FCS, 2 mM glutamine, 100 U/mI penicillin and 100 μg/ml streptomycin. The OVA-specific CTL line 4G3 (H-2 K^{b}-restricted, OVA-specific) was carried in RPMI 1640,10% heat inactivated FCS, 2 mM glutamine and 30 U/ml IL-2 (Genzyme, Cambridge, Mass.). Cells were split every 2-4 days and restimulated weekly with irradiated E.G7-OVA cells at 1:1 ratio. OVA peptide (H-2K^{b}-restricted, SIINFEKL, aa 257-264), and the control mut-1 peptide (H-2K^{b}-restricted, FEQNTAQP) were purchased from Research Genetics, (Huntsville, Alabama).

Chaperone Purification.

Chaperone proteins were purified from solid tumors as described by Wearsch and Nicchitta (1996) Prot. Express. Purif. 7:114-121. Tumors were established in either C57BL/6 (B16/F10.9 melanoma) or SCID (EL4, E.G7-OVA thymoma) mice. Solid tumors were harvested, a microsomal, endoplasmic reticulum (ER) enriched subfraction prepared, and the ER chaperones GRP94 and calreticulin purified to homogeneity from the microsomal fraction by selective detergent release, sequential Mono-Q™ 10/10 anion exchange, SUPERDEX™ 26/60 gel filtration chromatography (Pharmacia Biotech, Piscataway, N.J.) and centrifugal ultrafiltration (Amicon, Beverly, Mass.), as described by Wearsch and Nicchitta (1996) Prot Express. Purif. 7:114-121.

The cytosol fraction obtained upon subfractionation of the tumor homogenate was used to purify Hsp90 and Hsp70. The cytosol fraction was initially subjected to a 50-70% ammonium sulfate fractionation. Protein precipitated at 70% ammonium sulfate was resuspended in buffer A (110 mM KOAc, 20 mM NaCl, 20 mM K-HEPES, 0.5 mM PMSF), centrifuged for 20 min at 4° C. (100,000× g), to remove aggregated material, and the soluble fraction fractionated by preparative gel filtration on a SUPERDEX™ 26/60 column equilibrated in buffer A, at a flow rate of 1.5 ml/min. Fractions containing Hsp70 or Hsp90 were identified by SDS-PAGE, pooled, and sequentially chromatographed on MonoQ™ 10/10 and SUPERDEX™ 26/60 as described by Wearsch and Nicchitta (1996) Prot Express. Purif. 7:114-121.

BiP, ERp72, and PDI fractions arising from MonoQ™ fractionation of lumenal protein extracts, as well as Hsp70 and Hsp90 fractions eluting from the final SUPERDEX™ 26/60 step, were adjusted to 10 mM sodium phosphate, pH 6.8, loaded onto 2.5 ml hydroxylapatite columns (Bio-Rad HTP, Hercules, Calif.), and eluted with a 25 ml gradient of 10-300 mM sodium phosphate, pH 6.8. By SDS-PAGE, the purity of the Hsp90 and Hsp70 fractions was determined to be >95%. Protein purity was assessed by one and two dimensional SDS-PAGE.

Peptide Extraction and Analysis.

Calreticulin-associated peptides were extracted from 1 mg (21.7 nmol) of purified porcine by denaturation for 30 minutes (min) at room temperature (RT) in the presence of guanidinium chloride/1% trifluoracetic acid (TFA). The acid soluble fraction was separated from intact calreticulin by centrifugal ultrafiltration, using acid-washed CENTRICON™-10 filtration units. The low molecular weight calreticulin-derived peptide fraction was subsequently bound to a pre-washed Sep-Pak™ Cl 8 unit, washed extensively with 1%TFA, and eluted in 80% acetonitrile, 0.1% TFA. The acetonitrile eluate was dried by vacuum centrifugation and fractions either resuspended in 0.2 M Na phosphate, pH 7.2 and subject to reductive methylation with [³H] sodium borohydride, as described by Tack et al. (1980) J. Biol. Chem. 255:8842-8847, or subject to acid hydrolysis in vacuo, and the amino acid content determined by quantitative amino acid analysis.

Quantitative amino acid analysis was performed by the Duke University Medical Center Protein Sequencing Facility, a core facility of the Duke University Comprehensive Cancer Center, Durham, N.C. As a consequence of acid hydrolysis, tryptophan content cannot be determined, and asparagine and glutamine are hydrolyzed to aspartate and glutamate. In reductive methylation studies, the radiolabeled pool was fractionated on SEPHADEX™ G-10, to remove unincorporated isotope, concentrated, and analyzed on a Pharmacia SUPERDEX™ peptide column. Sample absorbance at 280 nm was continuously monitored. Fractions were collected and [³H] content determined by liquid scintillation chromatography.

Induction of Antigen-specific CTL In vivo.

Splenic dendritic cells (DC) or bone marrow precursor derived DC were generated as described by Mitchell et al. (1998) Eur. J. Immunol. 28:1923-1933 and Nair et al. (1997) Int. J. Cancer 70:706-715. DC (day 9 precursor-derived DC) or splenic DC were pulsed with heat shock proteins in the presence of the lipid, DOTAP (Boehringer-Mannheim, Indianapolis, Ind.) or DMRIE (Vical, San Diego, Calif.). Heat shock proteins (in 100 μl Opti-MEM) and DMRIE (in 100 μl Opti-MEM) were mixed at room temperature (RT) for 15 minutes. The complex was added to the DC in a total volume of 1 ml and incubated at 37° C. in a water-bath for 20-30 min. Alternatively, immature DC (day 7 precursor-derived) were pulsed with heat shock proteins in the absence of DMRIE for 48 h. Naive, syngeneic mice were immunized intravenously with 5×10⁵ precursor-derived DC or 1×10⁶ spleen-derived DC per mouse in 200 μl PBS.

Splenocytes were harvested after 10 days and depleted of red blood cells with ammonium chloride/Tris buffer. 1.0×10⁷ splenocytes were cultured with 5×10⁵ irradiated stimulator cells (E.G7-OVA cells irradiated at 20,000 rads, or F10.9 cells pretreated with IFN-γ and irradiated at 7500 rads) in 5 ml of IMDM with 10% FCS, 1 mM sodium pyruvate, 100 μU/ml penicillin, 100 μg/ml streptomycin and 5×10⁻⁵ M β-mercaptoethanol per well in a 6-well tissue culture plate. Cells were cultured for 5 days at 37° C. and 5% CO₂. Effectors were harvested on day 5 on Histopaque 1083 gradient prior to use in a CTL assay.

In vitro Cytotoxicity Assay.

5-10×10⁶ target cells were labeled with europium for 20 minutes at 4° C. 10⁴ europium-labeled targets and serial dilutions of effector cells at varying E:T were incubated in 200 μl of complete RPMI 1640. The plates were centrifuged at 500 g for 3 minutes and incubated at 37° C. for 4 hours. 50 μl of the supernatant was harvested and europium release was measured by time resolved fluorescence (Mitchell et al. (1998) Eur. J. Immunol. 28:1923-1933 and Nair et al. (1997) Int. J. Cancer. 70:706-715). Specific cytotoxic activity was determined using the formula: % specific release={(experimental release−spontaneous release)/(total release−spontaneous release)}×100. Spontaneous release of the target cells was less than 25% of total release by detergent in all assays. Standard errors of the means of triplicate cultures was less than 5%.

### Example 1

### Chaperone Purification and Peptide Extraction

A highly enriched ER microsome fraction was prepared from tissue homogenates by differential centrifugation and the lumenal protein components subsequently isolated from the microsomes by partial detergent extraction as described by Wearsch and Nicchitta (1996) Prot. Express. Purif. 7:114-121. Peripheral and integral ER membrane proteins remain in association with the detergent permeabilized membranes and can thus be efficiently segregated from the lumenal protein extract by centrifugation. The supernatant fraction resulting from this step contains five major polypeptides, GRP94(gp96), BiP, ERp72, protein disulfide isomerase (PDI) and calreticulin. In the final stage of the purification, calreticulin and GRP94 undergo gel filtration chromatography and centrifugal ultrafiltration (Wearsch and Nicchitta (1996) Prot. Express. Purif. 7:114-121).

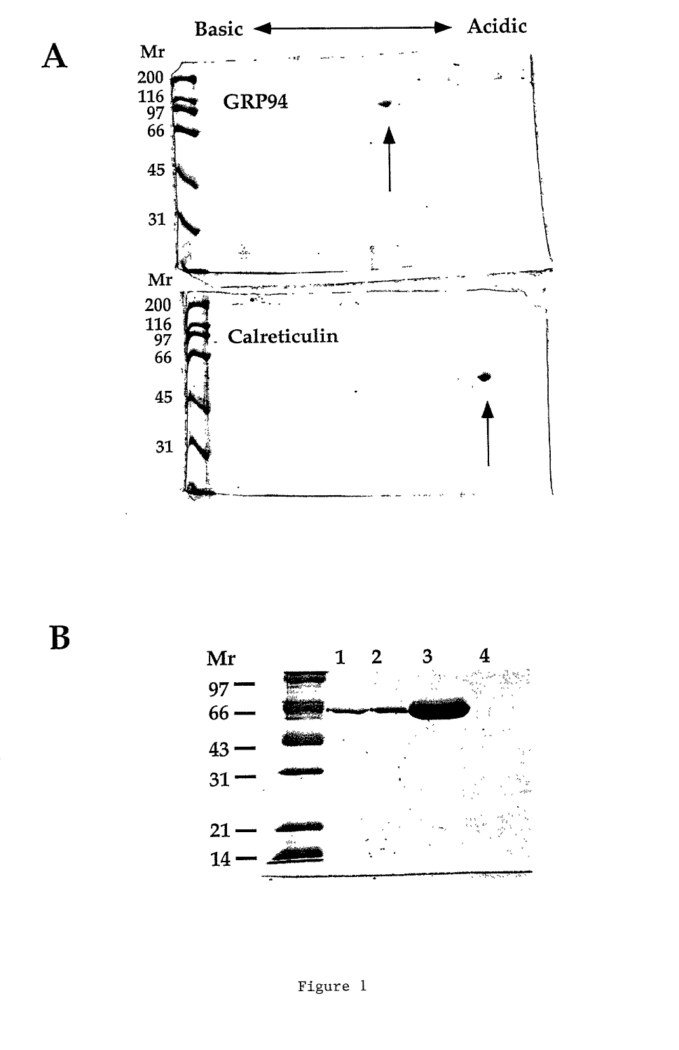

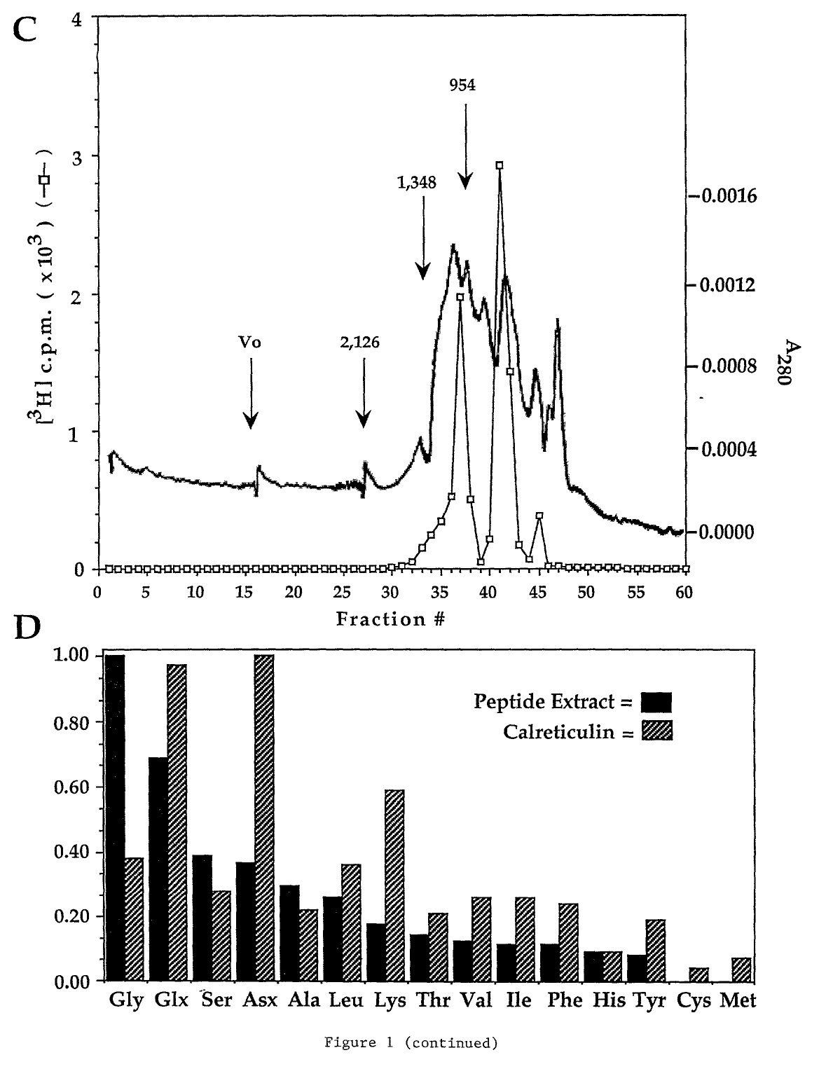

These procedures, in addition to yielding homogeneous preparations of the two proteins, were performed to eliminate circumstantial interactions between eitherofthe two chaperone proteins and low molecularweight peptide substrates. To assess the purity of the calreticulin and GRP94 used in these studies, representative samples were analyzed by 2-D SDS-PAGE. As shown in FIG. 1A, both proteins are, by this criterion, homogeneous.

Procedures were developed to extract calreticulin-bound peptides, and the peptide-enriched fraction separated from intact calreticulin by centrifugal ultrafiltration. As depicted in FIG. 1B, lane 3, SDS-PAGE analysis of the ultrafiltration retentate indicates that the conditions used for peptide extraction do not yield detectable hydrolysis or degradation of calreticulin. Analysis of the filtrate by SDS-PAGE analysis of the filtrate (FIG. 1B, lane 4) similarly shows no evidence of degradation products. To assay for the presence of peptides in the filtered extract, amine-specific radiolabeling (reductive methylation), followed by analytical gel filtration, or quantitative amino acid analyses were performed. Depicted in FIG. 1C, is the gel filtration elution profile of a representative fraction. When monitored at 280 nm, a broad band of UV-absorbing material was observed at elution volumes corresponding to 600-1500 molecular weight. This range of elution volumes overlapped with the elution of the radiolabeled material. For the radiolabeled material, the leading edge of the initial primary peak encompassed elution volumes corresponding to 1000-1900 molecular weight.

In a paired analysis, an equivalent quantity of calreticulin was extracted, and the peptide-enriched fraction, corresponding to the elution profile depicted in FIG. 1C, subjected to acid hydrolysis and quantitative amino acid analysis. FIG. 1D details the amino acid composition of the eluted material. For comparative purposes, and to assess whether the low molecular weight peptide fraction was a general calreticulin degradation product, the relative amino acid composition of calreticulin is depicted. The most abundant amino acid present in the calreticulin eluate was glycine, with the relative enrichment of those amino acids comprising greater than 20% of the total following the order Gly>Glu/Gln>Ser>Asp/Asn>Ala>Leu. On the basis of quantitative amino acid analysis, and with the assumption of a mean average peptide molecular weight of 1000, approximately 200 pmol of total peptide was recovered from 10 nmol of calreticulin.

### Example 2

### Induction of In vivo CTL responses by calreticulin and GRP94

To determine by immunological criteria whether calreticulin co-purifies in association with host tissue specific peptides, the capacity of calreticulin to elicit CTL responses in vivo was investigated in two model systems, the B16/F10.9 melanoma and EL4/E.G7-OVA. These model systems are described by by Nair et al. (1997) Int. J. Cancer. 70:706-715; Boczkowski et al. (1996) J. Exp. Med. 184:465-472; and Porgador et al. (1989) J. Immunogenet. 16:291-303.

For experiments using the B16/F10. 9 model, the ER chaperones GRP94, BiP, ERp72, protein disulfide isomerase (PDI) and calreticulin were purified to homogeneity from an F10.9 tumor-derived microsomal fraction. Control proteins were purified from either a normal spleen-derived microsomal fraction or from a porcine pancreas rough ER fraction as described by Wearsch and Nicchitta (1996) Prot. Express. Purif. 7:114-121).

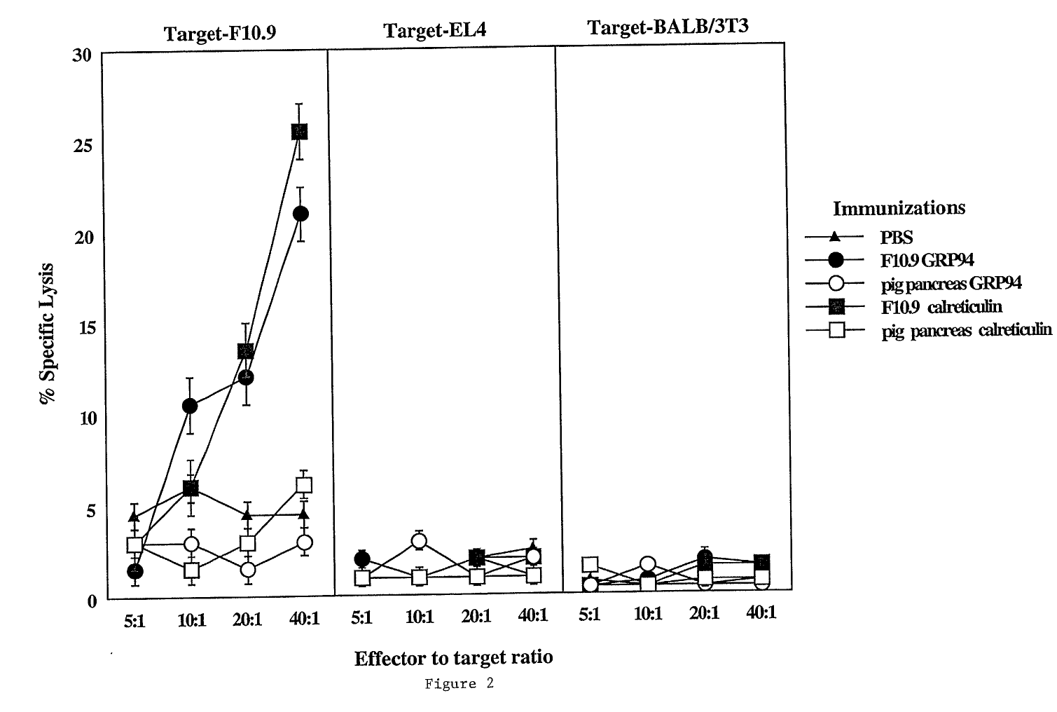

Mice were immunized twice intravenously at fourteen day intervals with 10 μg of hsp. A total of two immunizations were performed. Splenocytes were isolated from the immunized mice 10 days afterthe last immunization and were restimulated in vitro with irradiated IFN-γ pretreated F10.9 cells, and CTL activity assayed subsequently against F10.9 (H2-K^{b}), EL4 (H2-K^{b}), or BALB/3T3 (H2-K^{d}) cells. The results of a representative experiment are depicted in FIG. 2. Immunization with F10.9-derived calreticulin or GRP94 elicited a significant CTL response and the maximum level of CTL lysis observed was comparable for both proteins. That the observed CTL response, elicited by F10.9-derived calreticulin and GRP94, was specific for F10.9 cells was further substantiated by the fact that the control target cells, EL4 and BALB/3T3, exhibited no lysis (FIG. 2). Furthermore, no CTL responses were generated in mice immunized with DC pulsed with porcine calreticulin, porcine GRP94 or phosphate buffered saline. From these data, it is clear that tumor-derived calreticulin and GRP94 elicit an F10.9-specific CTL response.

### Example 3

### Determination of Chaperone-specificity: Induction of In vivo CTL Responses

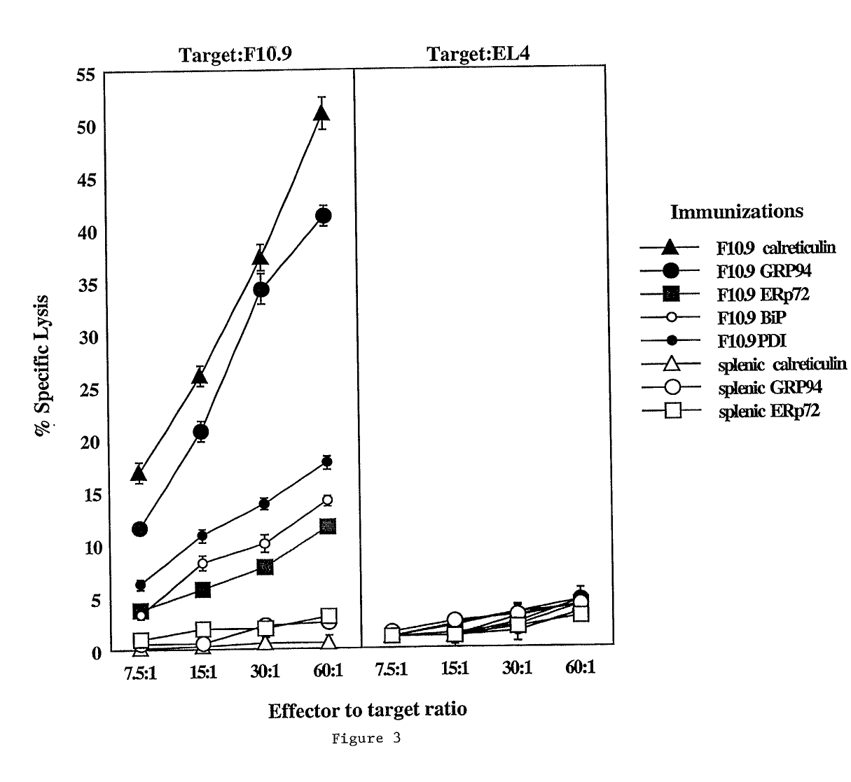

In this Example, the ability of the different ER chaperones to elicit a CTL response was examined. In these experiments, mice were immunized two times with precursor-derived DC pulsed, in the presence of a cationic lipid, with either mouse spleen-derived calreticulin, GRP94, or ERp72; or with F10.9 derived calreticulin, GRP94, ERp72, BiP or PDI. Splenocytes were restimulated ten days after the final immunization, and CTL activity assayed against F10.9 and EL4 cells (FIG. 3). Consistent with the data depicted in FIG. 2, immunization of mice with F10.9 calreticulin or GRP94-pulsed precursor-derived DC elicited a significant CTL response.

It is noteworthy that only low levels of CTL were generated by vaccination with tumor-derived PDI, ERp72 or BiP, though it is well established that these chaperones display peptide binding activity (Noiva et al. (1991) J. Biol. Chem. 266:19645-19649; Flynn et al. (1989) Science. 245:385-390). In these experiments, immunization with spleen derived calreticulin, GRP94, or ERp72 yielded little or no CTL. The control target EL4 showed no lysis. These results, as with those depicted in FIG. 2, demonstrate that immunization with calreticulin or GRP94 pulsed DC is sufficient to elicit a CTL response against antigens derived from the chaperone-host cell.

### Example 4

### Chaperone-dependent Elicitation of Peptide-specific CTL Responses

In the F10.9 system, immunization with tumor-derived calreticulin and GRP94 elicits a polyclonal CTL against an undefined set of tumor-associated antigens. Such results clearly identify these two proteins as immunogenic. Additional experiments were performed to determine if calreticulin and GRP94 associate with a known MHC class I peptide epitope, as defined by immunological criteria. Forthese experiments, the EL4/E.G7-OVA system was used. E.G7-OVA cells (EG7) are a clonal derivative of the EL4 tumor cell line (H-2b haplotype), and were selected for stable transfection with the chicken ovalbumin (OVA) cDNA (Moore et al. (1988) Cell. 54:777-785). In a C57BL/6 (H-2b) mouse background, expression of the chicken OVA gene yields the production of a single immunodominant OVA peptide epitope (aa 257-264) (Moore et al. (1988) Cell. 54:777-785.).

With respect to these studies, the EL4/E.G7-OVA experimental system offers two useful and interesting properties. One, chaperone elicited CTL responses against the OVA epitope can be assayed using OVA-specific clonal CTL lines. Two, the hypothesis that calreticulin and GRP94 bind unique and non-overlapping arrays of peptide substrates can be directly tested in determinations of shared EL4/E.G7-OVA CTL induction.

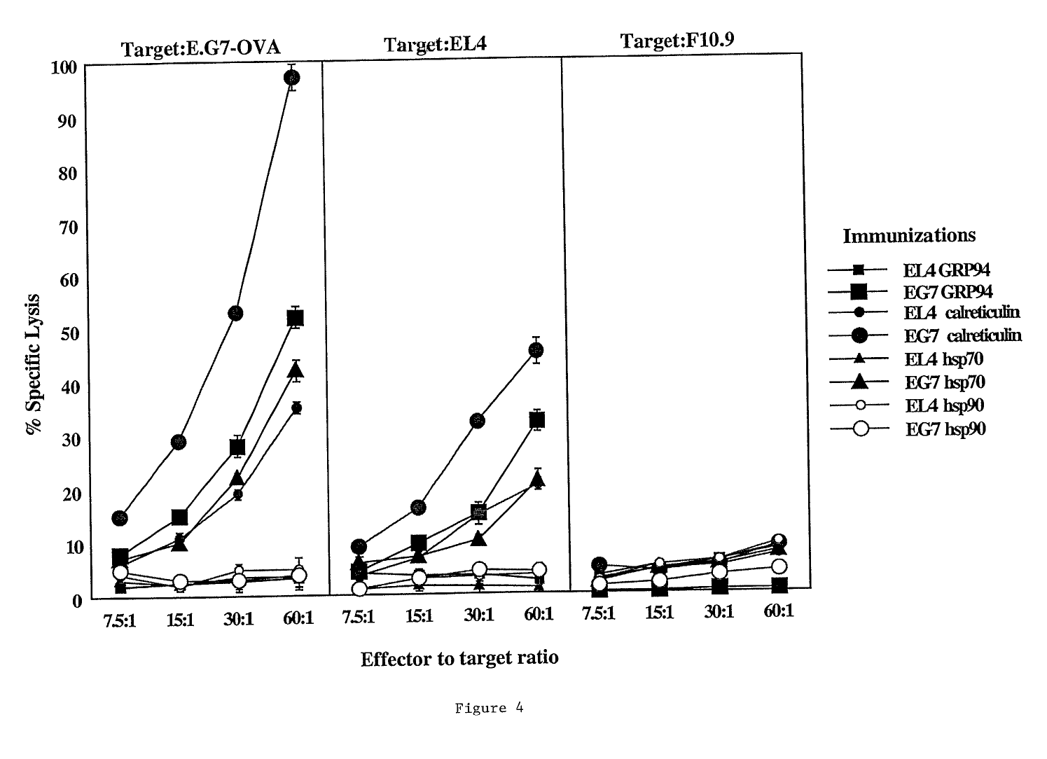

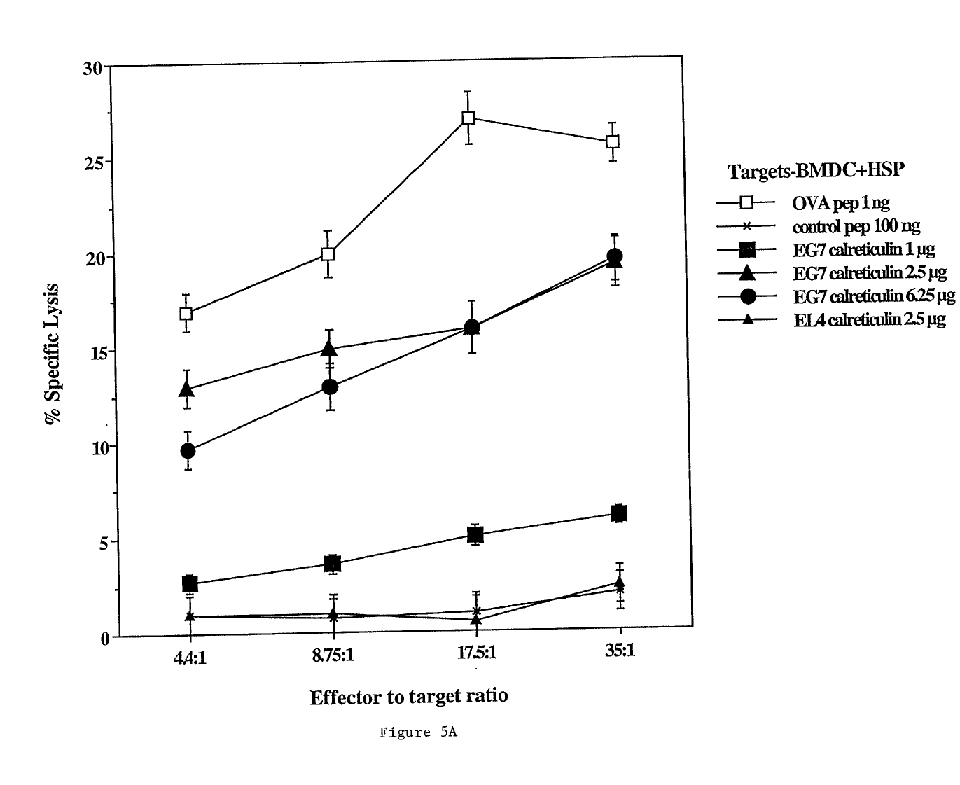

To prepare the relevant chaperone proteins, E.G7-OVA and EL4 tumors were established in SCID mice, a cytosol and microsome fraction prepared from excised tumors and calreticulin; and GRP94, Hsp90 and Hsp70 isolated from the relevant subcellular fractions. Splenic DC were pulsed in the presence of the cationic lipid DOTAP with either calreticulin, GRP94, Hsp90 or Hsp70, and mice subjected to a single vaccination, intravenously. CTL assays were then performed on splenocytes were isolated from immunized animals (FIG. 4).

As is evident in FIG. 4, E.G7-OVA-derived calreticulin and GRP94 elicted robust CTL responses against E.G7-OVA target cells. A substantial CTL response was also observed in the case of E.G7-OVA Hsp70 (FIG. 4). The data regarding GRP94 and Hsp70 are in agreement with previous studies demonstrating that GRP94, HSP70, and to lesser extent HSP90, when isolated from appropriate cells, prime antigen-specific CTL in vivo (Suto and Srivastava (1995) Science 269:1585-1588.; Tamura et al. (1997) Science 278:117-120).

Particularly noteworthy in the data depicted in FIG. 4 is the observation that immunization with EL4-derived calreticulin elicited CTL's against the E.G7-OVA target. Although applicants do not wish to be bound by a particular theory, it is believed that these data can be explained by the existence of a significant overlap in the spectrum of immunogenic peptides produced by the two closely related cells lines, at least a subset of which can stably associate with calreticulin.

When EL4 cells were used as targets (FIG. 4) a similar overall pattern was observed. These data were unexpected, as they indicated that E.G7-OVA-derived calreticulin and GRP94 elicited a more substantial CTL response to EL4 target cells, than that elicted by the EL4-derived proteins. Given that the relative immunogenicity of the different chaperone preparations is similar against both E.G7-OVA and EL4 target cells, it appears that the E.G7-OVA derived proteins are of higher relative antigenicity than those obtained from EL4. On the premise that the relative antigenicity of the chaperones is a direct function of the complement of bound peptides, these data suggest that the spectrum of immunogenic peptides present on the EL4 and E.G7-OVA chaperones displays significant similarities, as both elicit CTL responses against the related cell line, and significant differences, as the E.G7-OVA chaperones are more antigenic than those derived from EL4.

To alleviate concerns regarding CTL specificity, control experimentswith an unrelated target cell line (F10.9) were performed (FIG. 4). With F10.9 as the target cell, no significant CTL activity was observed in splenocyte preparations derived from animals immunized with any of the hsp preparations. These data substantiate the conclusion that the CTL activity observed against E.G7-OVA and EL4 target cells is specific, and thus, that the observed cross-cell reactivity is, at a fundamental level, a reflection of shared immunogenic epitopes co-purifying with the different chaperone protein preparations.

### Example 5

### Re-presentation of Calreticulin Associated Peptides

To better define the OVA specificity of the observed CTL responses, the capacity of immature, bone marrow-derived dendritic cells (BMDC) to present calreticulin-associated peptides, and be recognized for lysis by OVA-specific CTL, was investigated. Immature BMDC, as professional antigen presenting cells, process and present exogenous antigens on the class I pathway, and are thought to utilize this pathway for the activation of CD8+ CTL in vivo (Steinman, R. M. (1991) Annu. Rev. Immunol. 9:271-294; Matzinger, P. (1994) Annu. Rev. Immunol. 12:991-1045; Carbone et al. (1998) Immunol. Today. 19:103-109). Immature murine BMDC were pulsed with either the immunodominant ovalbumin peptide (SIINFEKL), a control peptide (mut-1), EL4 calreticulin, or E.G7-OVA calreticulin, and, following maturation, class I presentation of the ovalbumin epitope assayed by a CTL assay.

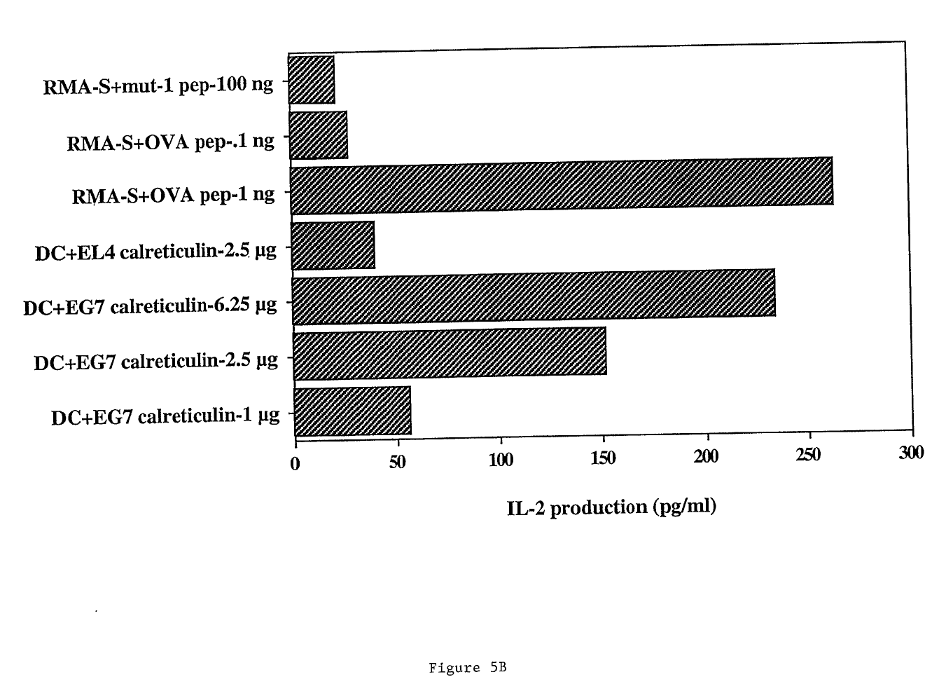

In these assays, calreticulin-pulsed BMDC served as target cells, and the OVA-specific CTL line 4G3 as effector cells. As shown in FIG. 5A, immature BMDC pulsed with E.G7-OVA-derived calreticulin present OVA for recognition and lysis by 4G3 CTL's, whereas no activity was observed for EL4 calreticulin. The specificity of this response is further supported by the data demonstrating that sensitization of the E.G7-OVA-calreticulin pulsed BMDC to lysis by 4G3 was dependent upon the concentration of E.G7-OVA calreticulin present in the media. As additional controls, it was observed that lysis could be elicited by the OVA peptide, whereas a 100-fold excess of the control peptide was without effect.

These observations were further expanded in the experiment depicted in FIG. 5B. In this experiment, presentation of the class I-restricted OVA epitope was assayed using a T cell hybridoma IL-2 secretion assay. In this assay, BMDC were cultured in the presence of E.G7-OVA or EL4-derived chaperone proteins, harvested, and assayed for their ability to stimulate IL-2 secretion from a class I restricted, OVA-specific T cell hybridoma (Mitchell et al. (1998) Eur. J. Immunol. 28:1923-1933). For comparative purposes, IL-2 secretion elicited in response to RMA-S cells pulsed with either control or OVA peptide was assayed. As depicted in FIG. 5B, the OVA-specificity of the assay was confirmed by data demonstrating that neither control peptide or EL4-derived calreticulin elicited IL-2 secretion, whereas OVA peptide elicited a dose dependent production of IL-2 (FIG. 5B). E.G7-OVA-derived calreticulin pulsed BMDC exhibited a dose-dependent stimulation of IL-2 secretion. These data clearly demonstrate that calreticulin-associated OVA peptide, or a structural precursor(s), was processed by BMDC and presented for recognition by the OVA-specific, class I restricted T cell hybridoma (Steinman, R. M. (1991) Annu. Rev. Immunol. 9:271-294; Matzinger, P. (1994) Annu. Rev. Immunol. 12:991-1045; Carbone et al. (1998) Immunol. Today. 19:103-109).

### Discussion of Examples

The results reported herein provide immunological and chemical evidence that calreticulin is a peptide binding protein. Furthermore, these data demonstrate that at least a subset of the peptides bound by calreticulin are appropriate ligands, or ligand precursors, for nascent MHC class I molecules. The immunological significance of these data is highlighted by the observation that pulsing of bone marrow-derived dendritic cells (BMDC) with soluble, exogenous calreticulin yielded presentation of calreticulin-derived peptides in association with BMDC class I molecules and lysis by an OVA-restricted CTL line. In addition to demonstrating that calreticulin and GRP94 can elicit CTL responses against components of their bound peptide pool, these data make evident the possibility that persistent release of calreticulin or GRP94 into the extracellular space, as might arise in chronic inflammation or tissue necrosis, may elicit a CTL response against the tissue comprising the site of chaperone release.

An additional aspect of calreticulin-based immunotherapeutic methods of the present invention concerns the diversity of bound antigenic peptides, a diversity likely reflective of the antigenic repertoire of the host cell. This aspect is particularly evident in the data presented in FIG. 4, in which the capacity of ER lumenal chaperones derived from EL4 and E.G7-OVA thymoma tumors to elicit CTL's directed against EL4 and E.G7-OVA was determined.

### REFERENCES

The references listed below as well as all references cited in the specification are incorporated herein by reference to the extent that they supplement, explain, provide a background for or teach methodology, techniques and/or compositions employed herein.

Arnold et al. (1995) J. Exp. Med. 182:885-889.

Bevan, M. J. (1995) J. Exp. Med. 182:639-641.

Blachere et al. (1997) J. Exp. Med. 186:1315-1322.

Blachere et al., 1993, J. Immunotherapy 14:352-356

Blachere et al. (1993) J. Immunotherapy 14:352-356.

Boczkowski et al. (1996) J. Exp. Med. 184:465-472.

Bodanszk et al. (1976) “Peptide Synthesis”, John Wiley & Sons, Second Ed.

Brawer et. al. (1992) J. Urol. 147:841-845.

Bumal (1988) Hybridoma 7(4):407-415)

Carbone et al. (1998) Immunol. Today. 19:103-109.

Catalona et al. (1993) JAMA 270:948-958

Demotz et al. (1989) Nature 343:682-684

Elliott et al. (1990) Nature 348:191-197

Estin et al. (1989) J. Natl. Cancer Inst. 81 (6):445-446

Falk et al. (1990) Nature 348:248-251

Falk et al. (1991) Nature 351:290-296

Fearon et al. (1988) Cancer Res. 48:2975-2980.

Fields et al. (1990) Int. J. Peptide Protein Res. 35:161-214.

Flynn et al. (1989) Science 245:385-390.

Freireich et al. (1966) Cancer Chemotherap. Rep. 50:219-244

Glasebrook et al. (1980) J. Exp. Med. 151:876

Hebert et al. (1996) EMBO J. 15:2961-2968.

Hebert et al. (1997) J. Cell Biol. 139:613-623.

Heike et al. (199) J. Immunotherapy 15:165-174

Henttu and Vihko (1989) Biochem. Biophys. Res. Comm. 160(2):903-910.

Inaba (1992) J. Exp. Med. 176:1693-1702.

Israeli et al. (1993) Cancer Res. 53:227-230.

Lammert et al. (1997) Eur. J. Immunol. 27:1685-1690.

Levy et al. (1991) Cell 67:265-274

Li et al. (1993) EMBO Journal 12:3143-3151

Matzinger, P. (1994) Annu. Rev. Immunol. 12:991-1045.

McOmie, J. (1973) “Protective Groups in Organic Chemistry”, Plenum Press, New York.

Meienhofer, J. (1983) “Hormonal Proteins and Peptides”, Vol.2, p. 46, Academic Press (New York).

Merrifield (1969) Adv Enzymol, 32:221-96.

Mitchell et al. (1998) Eur. J. Immunol. 28:1923-1933.

Moore et al. (1988) Cell. 54:777-785.

Nair et al. (1997) Int. J. Cancer. 70:706-715.

Nash et al. (1994) Mol. Cell. Biochem. 135:71-78.

Natali et al. (1987) Cancer 59:55-63

Nicchitta, C. V. (1998) Curr. Opin. Immunol. 10:103-109.

Noiva et al. (1991) J. Biol. Chem. 266:19645-19649.

Norrby (1985) Summary, in Vaccines 85, Lerner, et al. (eds.), Cold Spring Harbor Laboratory, Cold Spring Harbor, N.Y., pp. 388-389

Ortmann et al. (1997) Science 277:1306-1309.

Palladino et al. (1987) Cancer Res. 47:5074-5079

Perez and Walker (1990) J. Immunol. 142:3662-3667

Porgador et al. (1989) J. Immunogenet. 16:291-303.

Powis, S. J. (1997) Eur. J. Immunol. 27:2744-2747.

Robbins and Angell (1976) Basic Pathology, 2d Ed., W. B. Saunders Co., Philadelphia, pp. 68-79

Rotzsche et al. (1990) Science 249:283-287

Rotzsche et al. (1990) Nature 348:252-254

Sadasivan et al. (1996) Cell 5:103-114.

Sato et al. (1995) Clin. Immunol. Pathol. 74:35-43

Schroder et al. (1965) “The Peptides”, Vol. 1, Academic Press, New York.

Shirkey, H. C. (1965) JAMA 193:443

Solheim et al. (1997) J. Immunol. 158:2236-2241.

Spee and Neefjes (1997) Eur. J. Immunol. 27:2441-2449.

Spiro et al. (1996) J. Biol. Chem. 271:11588-11594.

Srivastava et al. (1998) Immunity 8:657-665.

Steinman, R. M. (1991) Annu. Rev. Immunol. 9:271-294.

Steward et al. (1969) “Solid Phase Peptide Synthesis”, W. H. Freeman Co., San Francisco.

Suto and Srivastava (1995) Science 269:1585-1588.

Tack et al. (1980) J. Biol. Chem. 255:8842-8847.

Tailer et al. (1990) Nucl. Acids Res. 18(16):4928.

Tamura et al. (1997) Science 278:117-120.

U.S. Pat. No. 4,244,946

U.S. Pat. No. 5,750,119

U.S. Pat. No. 5,837,251

U.S. Pat. No. 5,830,464

Van Bleek et al. (1990) Nature 348:213-216

Vassilakos et al. (1998) Biochem. 37:3480-3490.

Vijayasardahl et al. (1990) J. Exp. Med. 171(4):1375-1380

Walden and Eisen (1990) Proc. Natl. Acad. Sci. USA. 87:9015-9019.

Watts, C. (1997) Annu. Rev. Immunol. 15:821-850.

Wearsch and Nicchitta (1996) Prot. Express. Purif. 7:114-121.

Wearsch and Nicchitta (1997) J. Biol. Chem. 272:5152-5156.

WO 95/24923

WO 97/10000

WO 97/10002

WO 98/34641

Yu et al. (1991) Cancer Res. 51(2):468-475

Zimmeret al. (1993) Peptides 1992, pp. 393-394, ESCOM Science Publishers, B.V.

It will be understood that various details of the invention may be changed without departing from the scope of the invention. Furthermore, the foregoing description is for the purpose of illustration only, and not for the purpose of limitation—the invention being defined by the claims.

What is claimed is:

1. A method of eliciting an immune response in a vertebrate subject, the method comprising the step of administering to the vertebrate subject a composition comprising an amount of a purified complex comprising calreticulin bound to an antigenic molecule, whereby an immune response to the antigenic molecule is elicited in the vertebrate subject.

2. The method of claim 1, wherein the complex is administered in an amount ranging from about 0.1 to about 1000 micrograms.

3. The method of claim 2, wherein the complex is administered in an amount ranging from about 10 to about 600 micrograms.

4. The method of claim 1, wherein the complex is administered in an amount of less than about 50 micrograms.

5. The method of claim 4, wherein the complex is administered in an amount of ranging from about 5 to about 49 micrograms.

6. The method of claim 1, wherein the complex is administered in an amount of less than about 10 micrograms.

7. The method of claim 6, wherein the complex is administered in an amount ranging from about 0.1 to about 9.0 micrograms.

8. The method of claim 7, wherein the complex is administered in an amount ranging from about 0.5 to about 2.0 micrograms.

9. The method of claim 1, wherein the administering step is repeated at weekly intervals.

10. The method of claim 1, wherein said complex is administered intramuscularly, subcutaneously, intraperitoneally, intravenously, intradermally or mucosally.

11. The method of claim 1, wherein the vertebrate subject is a human.

12. The method of claim 1, further comprising administering to the vertebrate subject an effective amount of a biological response modifier selected from the group consisting of interferon-α, interferon-γ, interleukin-2, interleukin-4, interleukin-6, tumor necrosis factor and combinations thereof.

13. A method of treating or preventing a type of cancer in a vertebrate subject, comprising administering to the vertebrate subject a composition comprising a therapeutically or prophylactically effective amount of a purified complex, said complex comprising calreticulin bound to an antigenic molecule specific to said type of cancer.

14. The method of claim 13, wherein the complex is administered in an amount ranging from about 0.1 to about 1000 micrograms.

15. The method of claim 14, wherein the complex is administered in an amount ranging from about 10 to about 600 micrograms.

16. The method of claim 13, wherein the complex is administered in an amount of less than about 50 micrograms.

17. The method of claim 16, wherein the complex is administered in an amount of ranging from about 5 to about 49 micrograms.

18. The method of claim 13, wherein the complex is administered in an amount of less than about 10 micrograms.

19. The method of claim 18, wherein the complex is administered in an amount ranging from about 0.1 to about 9.0 micrograms.

20. The method of claim 19, wherein the complex is administered in an amount ranging from about 0.5 to about 2.0 micrograms.

21. The method of claim 13, wherein said administering step is repeated at weekly intervals.

22. The method of claim 13, wherein said complex is administered intramuscularly, subcutaneously, intraperitoneally, intravenously, intradermally or mucosally.

23. The method of claim 13, wherein the vertebrate subject is a human.

24. The method of claim 13, wherein the complex of calreticulin and antigenic molecule is produced in vitro.

25. The method of claim 13, wherein the antigenic molecule is an exogenous antigenic peptide.

26. The method of claim 13, wherein the antigenic molecule is a peptide with which the calreticulin is endogenously associated in vivo.

27. The method of claim 13, wherein the complex is isolated from cancerous tissue.

28. The method of claim 13, wherein the cancerous tissue is from the vertebrate subject.

29. The method of claim 13, wherein the complex is obtained from tissue of said type of cancer.

30. The method of claim 13, wherein the complex is isolated from cancerous tissue autologous to the vertebrate subject.

31. The method of claim 13, wherein the complex is isolated from cancerous tissue allogeneic to the individual.

32. The method of claim 13, wherein the complex is obtained from a tumor cell line of said type of cancer.

33. The method of claim 13, wherein said type of cancer comprises a sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenströom's macroglobulinemia, and heavy chain disease.

34. The method of claim 13, further comprising administering to the vertebrate subject an effective amount of a biological response modifier selected from the group consisting of interferon-α, interferon-γ, interleukin-2, interleukin-4, interleukin-6, tumor necrosis factor and combinations thereof.

35. A method of treating or preventing an infectious disease in a vertebrate subject, the method comprising administering a therapeutically or prophylactically effective amount of a purified complex, said complex comprising calreticulin bound to an antigenic molecule specific to said infectious disease.

36. The method of claim 35, wherein the complex is administered in an amount ranging from about 0.1 to about 1000 micrograms.

37. The method of claim 36, wherein the complex is administered in an amount ranging from about 10 to about 600 micrograms.

38. The method of claim 35, wherein the complex is administered in an amount of less than about 50 micrograms.

39. The method of claim 38, wherein the complex is administered in an amount of ranging from about 5 to about 49 micrograms.

40. The method of claim 35, wherein the complex is administered in an amount of less than about 10 micrograms.

41. The method of claim 40, wherein the complex is administered in an amount ranging from about 0.1 to about 9.0 micrograms.

42. The method of claim 41, wherein the complex is administered in an amount ranging from about 0.5 to about 2.0 micrograms.

43. The method of claim 35, wherein said administering step is repeated at weekly intervals.

44. The method of claim 35, wherein said complex is administered intramuscularly, subcutaneously, intraperitoneally, intravenously, intradermally or mucosally.

45. The method of claim 35, wherein the vertebrate subject is a human.

46. The method of claim 35, wherein the complex of calreticulin and antigenic molecule is produced in vitro.

47. The method of claim 35, wherein the antigenic molecule is an antigenic peptide that is present in a eukaryotic cell infected with a pathogen which cause said infectious disease but not present in said eukaryotic cell when said eukaryotic cell is not infected with said pathogen.

48. The method of claim 35, wherein said infectious disease is caused by a pathogen selected from the group consisting of viruses, bacteria, fungi, protozoa and parasites.

49. The method of claim 48, wherein said viral pathogen is selected from the group consisting of hepatitis type A, hepatitis type B, hepatitis type C, influenza, varicella, adenovirus, herpes simplex type I (HSV-I), herpes simplex type II (HSV-I), rinderpest, rhinovirus, echovirus, rotavirus, respiratory syncytial virus (RSV), papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

50. The method of claim 48, wherein said bacterial pathogen is selected from the group consisting of Mycobacteria, Rickeffsia, Mycoplasma, Neisseria and Legionella.

51. The method of claim 48, wherein said protozoal pathogen is selected from the group consisting of Leishmania, Kokzidioa, and Trypanosoma.

52. The method of claim 48, wherein said protozoal pathogen is selected from the group consisting of Chiamydia and Rickettsia.

53. The method of claim 35, further comprising administering to the vertebrate subject an effective amount of a biological response modifier selected from the group consisting of interferon-α, interferon-γ, interleukin-2, interleukin-4, interleukin-6, tumor necrosis factor and combinations thereof.

54. A purified and isolated complex comprising calreticulin non-covalently bound to an antigenic molecule.

55. A pharmaceutical composition comprising an immunogenic amount of purified complex of claim 54 and a pharmaceutically acceptable carrier.

56. The composition of claim 55, wherein the complex is present in an amount ranging from about 0.1 to about 1000 micrograms.

57. The composition of claim 56, wherein the complex is present in an amount ranging from about 10 to about 600 micrograms.

58. The composition of claim 55, wherein the complex is present in an amount of less than about 50 micrograms.

59. The composition of claim 58, wherein the complex is present in an amount of ranging from about 5 to about 49 micrograms.

60. The composition of claim 55, wherein the complex is present in an amount of less than about 10 micrograms.

61. The composition of claim 60, wherein the complex is present in an amount ranging from about 0.1 to about 9.0 micrograms.

62. The composition of claim 61, wherein the complex is present in an amount ranging from about 0.5 to about 2.0 micrograms.

63. The composition of claim 55, wherein the complex of calreticulin and antigenic molecule is produced in vitro.

64. The composition of claim 55, wherein the antigenic molecule is a peptide with which the calreticulin is endogenously associated in vivo.

65. The composition of claim 55, wherein the complex is isolated from a cell of a type of cancer.

66. The composition of claim 65, wherein the cell from the type of cancer is isolated from a vertebrate subject.

67. The composition of claim 66, wherein the cell from the type of cancer is isolated from cancerous tissue autologous to a vertebrate subject to be treated with the composition.

68. The composition of claim 66, wherein the cell from the type of cancer is isolated from cancerous tissue allogeneic to a vertebrate subject to be treated with the composition.

69. The composition of claim 65, wherein the cell is obtained from a tumor cell line of said type of cancer.

70. The composition of claim 65, wherein said type of cancer comprises a sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenströom's macroglobulinemia, and heavy chain disease.

71. The composition of claim 55, wherein the complex of calreticulin and antigenic molecule is produced in vitro.

72. The composition of claim 55, wherein the antigenic molecule is an antigen of a pathogen.

73. The composition of claim 72, wherein said pathogen is selected from the group consisting of viruses, bacteria, fungi, protozoa and parasites.

74. The composition of claim 73, wherein said viral pathogen is selected from the group consisting of hepatitis type A, hepatitis type B, hepatitis type C, influenza, varicelIa, adenovirus, herpes simplex type I (HSV-I), herpes simplextype II (HSV-I), rinderpest, rhinovirus, echovirus, rotavirus, respiratory syncytial virus (RSV), papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

75. The composition of claim 73, wherein said bacterial pathogen is selected from the group consisting of Mycobacteria, Rickeffsia, Mycoplasma, Neisseria and Legionella.

76. The composition of claim 73, wherein said protozoal pathogen is selected from the group consisting of Leishmania, Kokzidioa, and Trypanosoma.

77. The composition of claim 73, wherein said protozoal pathogen is selected from the group consisting of Chlamydia and Rickettsia.

78. The composition of claim 55, further comprising an effective amount of a biological response modifier selected from the group consisting of interferon-α, interferon-γ, interleukin-2, interleukin-4, interleukin-6, tumor necrosis factor and combinations thereof.

79. A method of eliciting an immune response in a vertebrate subject, the method comprising the step of administering to the vertebrate subject an immunogenic amount of sensitized antigen presenting cells, wherein the antigen presenting cells have been sensitized in vitro with a complex comprising calreticulin bound to an antigenic molecule, whereby an immune response to the antigenic molecule is elicited in the vertebrate subject.

80. The method of claim 79, wherein the antigen presenting cells are selected from the group consisting of macrophage, dendritic cells, B cells and combinations thereof.

81. The method of claim 79, wherein about 10⁶ to about 10¹² antigen presenting cells are administered.

82. The method of claim 79, wherein the administering step is repeated at weekly intervals.

83. The method of claim 79, wherein said sensitized antigen presenting cells are administered intramuscularly, subcutaneously, intraperitoneally, mucosally, intradermally or intravenously.

84. The method of claim 79, wherein the vertebrate subject is a human.

85. The method of claim 79, further comprising administering to the vertebrate subject an effective amount of a biological response modifier selected from the group consisting of interferon-α, interferon-γ, interleukin-2, interleukin-4, interleukin-6, tumor necrosis factor and combinations thereof.

86. A method of treating or preventing a type of cancer in a vertebrate subject, comprising administering to the vertebrate subject an therapeutically or prophylactically effective amount of sensitized antigen presenting cells, wherein the antigen presenting cells have been sensitized in vitro with a complex comprising calreticulin bound to an antigenic molecule specific to said type of cancer.

87. The method of claim 86, wherein the antigen presenting cells are selected from the group consisting of macrophage, dendritic cells, B cells and combinations thereof.

88. The method of claim 86, wherein about 10⁶ to about 10¹² antigen presenting cells are administered.

89. The method of claim 86, wherein the administering step is repeated at weekly intervals.

90. The method of claim 86, wherein said sensitized antigen presenting cells are administered intramuscularly, subcutaneously, intraperitoneally, mucosally, intradermally or intravenously.

91. The method of claim 86, wherein the vertebrate subject is a human.

92. The method of claim 86, wherein the complex of calreticulin and antigenic molecule is produced in vitro.

93. The method of claim 86, wherein the antigenic molecule is an exogenous antigenic peptide.

94. The method of claim 86, wherein the antigenic molecule is a peptide with which the calreticulin is endogenously associated in vivo.

95. The method of claim 86, wherein the complex is isolated from cancerous tissue.

96. The method of claim 86, wherein the cancerous tissue is from the vertebrate subject.

97. The method of claim 86, wherein the complex is obtained from tissue of said type of cancer.

98. The method of claim 86, wherein the complex is isolated from cancerous tissue autologous to the vertebrate subject.

99. The method of claim 86, wherein the complex is isolated from cancerous tissue allogeneic to the individual.

100. The method of claim 86, wherein the complex is obtained from a tumor cell line of said type of cancer.

101. The method of claim 86, wherein said type of cancer comprises a sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladdercarcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenströom's macroglobulinemia, and heavy chain disease.

102. The method of claim 86, further comprising administering to the vertebrate subject an effective amount of a biological response modifier selected from the group consisting of interferon-α, interferon-γ, interleukin-2, interleukin-4, interleukin-6, tumor necrosis factor and combinations thereof.

103. A method of treating or preventing an infectious disease in a vertebrate subject, the method comprising administering a therapeutically or prophylactically effective amount of sensitized antigen presenting cells, wherein the antigen presenting cells have been sensitized in vitro with a complex comprising calreticulin bound to an antigenic molecule specific to said infectious disease.

104. The method of claim 103, wherein the antigen presenting cells are selected from the group consisting of macrophage, dendritic cells, B cells and combinations thereof.

105. The method of claim 103, wherein about 10⁶ to about 10¹² antigen presenting cells are administered.

106. The method of claim 103, wherein the administering step is repeated at weekly intervals.

107. The method of claim 103, wherein said sensitized antigen presenting cells are administered intramuscularly, subcutaneously, intraperitoneally, mucosally, intradermally or intravenously.

108. The method of claim 103, wherein the vertebrate subject is a human.

109. The method of claim 103, wherein the complex of calreticulin and antigenic molecule is produced in vitro.

110. The method of claim 103, wherein the antigenic molecule is an antigenic peptide that is present in a eukaryotic cell infected with a pathogen which cause said infectious disease but not present in said eukaryotic cell when said eukaryotic cell is not infected with said pathogen.

111. The method of claim 103, wherein said infectious disease is caused by a pathogen selected from the group consisting of viruses, bacteria, fungi, protozoa and parasites.

112. The method of claim 111, wherein said viral pathogen is selected from the group consisting of hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus (RSV), papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

113. The method of claim 111, wherein said bacterial pathogen is selected from the group consisting of Mycobacteria, Rickettsia, Mycoplasma, Neisseria and Legionella.

114. The method of claim 111, wherein said protozoal pathogen is selected from the group consisting of Leishmania, Kokzidioa, and Trypanosoma.

115. The method of claim 111, wherein said protozoal pathogen is selected from the group consisting of Chlamydia and Rickettsia.

116. The method of claim 103, further comprising administering to the vertebrate subject an effective amount of a biological response modifier selected from the group consisting of interferon-α, interferon-γ, interleukin-2, interleukin-4, interleukin-6, tumor necrosis factor and combinations thereof.

117. A pharmaceutical composition comprising an immunogenic amount of sensitized antigen presenting cells, wherein the antigen presenting cells have been sensitized in vitro with a complex comprising calreticulin bound to an antigenic molecule, and a pharmaceutically acceptable carrier.

118. The composition of claim 117, wherein the antigen presenting cells are selected from the group consisting of macrophage, dendritic cells and combinations thereof.

119. The composition of claim 117, further comprising about 10⁶ to about 10¹² antigen presenting cells.

120. The composition of claim 117, wherein the complex of calreticulin and antigenic molecule is produced in vitro.

121. The composition of claim 117, wherein the antigenic molecule is a peptide with which the calreticulin is endogenously associated in vivo.

122. The composition of claim 117, wherein the complex is isolated from a cell of a type of cancer.

123. The composition of claim 122, wherein the cell from the type of cancer is isolated from a vertebrate subject.

124. The composition of claim 123, wherein the cell from the type of cancer is isolated from cancerous tissue autologous to a vertebrate subject to be treated with the composition.

125. The composition of claim 123, wherein the cell from the type of cancer is isolated from cancerous tissue allogeneic to a vertebrate subject to be treated with the composition.

126. The composition of claim 122, wherein the cell is obtained from a tumor cell line of said type of cancer.

127. The composition of claim 122, wherein said type of cancer comprises a sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenströom's macroglobulinemia, and heavy chain disease.

128. The composition of claim 117, wherein the antigenic molecule is an antigen of a pathogen.

129. The composition of claim 128, wherein said pathogen is selected from the group consisting of viruses, bacteria, fungi, protozoa and parasites.

130. The composition of claim 129, wherein said viral pathogen is selected from the group consisting of hepatitis type A, hepatitis type B, hepatitis type C, influenza, varicella, adenovirus, herpes simplex type I (HSV-I), herpes simplextype II (HSV-I), rinderpest, rhinovirus, echovirus, rotavirus, respiratory syncytial virus (RSV), papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

131. The composition of claim 129, wherein said bacterial pathogen is selected from the group consisting of Mycobacteria, Rickettsia, Mycoplasma, Neisseria and Legionella.

132. The composition of claim 129, wherein said protozoa pathogen is selected from the group consisting of Leishmania, Kokzidioa, and Trypanosoma.

133. The composition of claim 129, wherein said protozoal pathogen is selected from the group consisting of Chlamydia and Rickeffsia.

134. The composition of claim 117, further comprising an effective amount of a biological response modifier selected from the group consisting of interferon-α, interferon-γ, interleukin-2, interleukin-4, interleukin-6, tumor necrosis factor and combinations thereof.

135. A method for preparing an immuogenic composition for inducing an immune response in a vertebrate subject, the method comprising:
(a) harvesting from a eukaryotic cell an immunogenic complex comprising calreticulin non-covalently bound to an antigenic molecule, said complex, when administered to said vertebrate subject being operative at initiating an immune response in said vertebrate subject; and
(b) combining said complex with pharmaceutically acceptable carrier.

136. The method of claim 135, wherein the antigenic molecule is a peptide with which the calreticulin is endogenously associated in vivo.

137. The method of claim 135, wherein the complex is harvested from a cell of a type of cancer.

138. The method of claim 137, wherein the cell from the type of cancer is isolated from a vertebrate subject.

139. The method of claim 138, wherein the cell from the type of cancer is isolated from cancerous tissue autologous to a vertebrate subject to be treated with the immunogenic composition.

140. The method of claim 138, wherein the cell from the type of cancer is isolated from cancerous tissue allogeneic to a vertebrate subject to be treated with the immunogenic composition.

141. The method of claim 137, wherein the cell is obtained from a tumor cell line of said type of cancer.

142. The method of claim 135, wherein the eukaryotic cell has been transfected with a nucleic acid construct encoding the antigenic molecule, whereby the antigenic molecule is expressed in the eukaryotic cell.

143. The method of claim 135, wherein the eukaryotic cell comprises a cell infected with a pathogen.

144. The method of claim 143, wherein the antigenic molecule is an antigenic peptide that is present in said eukaryotic cell infected with said pathogen but not present in said eukaryotic cell when said eukaryotic cell is not infected with said pathogen.

145. A method for preparing an immunogenic composition for inducing an immune response in a vertebrate subject, the method comprising:
(a) reconstituting in vitro an antigenic molecule and calreticulin molecule to thereby produce an immunogenic complex comprising calreticulin non-covalently bound to an antigenic molecule, said complex, when administered to said vertebrate subject being operative at initiating an immune response in said vertebrate subject; and
(b) combining said complex with pharmaceutically acceptable carrier.

146. The method of claim 145, wherein the antigenic molecule is a peptide with which the calreticulin is endogenously associated in vivo.

147. The method of claim 146, wherein the antigenic molecule is a cancer antigen.

148. The method of claim 145, wherein the antigenic molecule is an exogenous antigenic peptide.

149. The method of claim 148, wherein the antigen molecule is peptide from a pathogen.

150. The method of claim 145, wherein the calreticulin and the antigenic molecule are admixed in a buffer comprising 20 mM sodium phosphate, pH 7.2, 350 mM NaCl, 3 mM MgCl2 and 1 mM phenyl methyl sulfonyl fluoride (PMSF).

151. A product produced by the methods of any of claims 135-150.

152. A method for preparing an immunogenic composition for inducing an immune response in a vertebrate subject, the method comprising:
(a) sensitizing antigen presenting cells in vitro with a complex comprising calreticulin non-covalently bound to an antigenic molecule; and
(b) combining said at least one sensitized antigen presenting cell with pharmaceutically acceptable carrier.

153. The method of claim 152, wherein the antigenic molecule is a peptide with which the calreticulin is endogenously associated in vivo.

154. The method of claim 153, wherein the antigenic molecule is a cancer antigen.

155. The method of claim 152, wherein the antigenic molecule is an exogenous antigenic peptide.

156. The method of claim 155, wherein the antigen molecule is a peptide from a pathogen.

157. A product produced by the methods of any of claims 152-156.
